(19) United States
(12) Patent Application Publication
(10) Pub. No.: US 2008/0221014 A1
(43) Pub. Date: Sep. 11, 2008

(54) Method of Diagnosing and Treating Glioma

(75) Inventors: Heidi S. Phillips, Palo Alto, CA (US); Liliana Soroceanu, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/946,449

(22) Filed: Nov. 28, 2007

Related U.S. Application Data
(60) Provisional application No. 60/867,761, filed on Nov. 29, 2006.

(51) Int. Cl. A61K 38/02; C12N 5/00; A61K 31/7052; A61P 35/00; C12Q 1/68

(52) U.S. Cl. 514/2; 435/375; 514/44; 435/6

(57) ABSTRACT

The present invention is directed to methods and compositions for the diagnosis, prognosis and treatment of glioma in mammals.

### CROSS-REFERENCE TO RELATED APPLICATIONS

This application claims priority under 35 U.S.C. § 119(e) to U.S. Ser. No. 60/867,761, filed Nov. 29, 2006, which is incorporated by reference in its entirety.

### FIELD OF THE INVENTION

The present invention is directed to methods of diagnosing, prognosing and treating glioma.

### BACKGROUND OF THE INVENTION

Gliomas are the most common type of primary brain tumors and are typically associated with grave prognosis. High-grade astrocytomas, which include glioblastoma multiformans (GBM) and anaplastic astrocytoma (AA), are the most common intrinsic brain tumors in adults. While there has been progress in understanding the molecular genetics of high-grade astrocytomas, the cell type(s) of origin are still uncertain and the molecular determinants of disease aggressiveness are not well understood. A better understanding of the cellular origin and molecular pathogenesis of these tumors may identify new targets for treatment of these neoplasms that are nearly uniformly fatal.

The grading of tumors is often critical to an accurate diagnosis and prognosis of disease progression, and brain cancer is no exception. Decades of experience have lead to a system of diagnosis of gliomas based on histology. Gliomas are histologically defined by whether they exhibit primarily astrocytic or oligodendroglial morphology. Gliomas are graded by cellularity, nuclear atypia, necrosis, mitotic figures, and micro-vascular proliferation—all features associated with biologically aggressive behavior. This system of diagnosis has been developed over decades of clinical experience with gliomas and has now become the cornerstone of neuro-oncology. Kleihues, P. et al., World Health Organization (“WHO”) classification of tumors, Cancer 88: 2887 (2000). The WHO classification scheme of astrocytic gliomas is divided into four (4) grades. Less malignant tumors fall under Grade I (pilocytic astrocytoma) and Grade II (astrocytic glioma), whereas the more malignant tumors are designated Grade III (anaplastic astrocytoma) and Grade IV (GBM). Oligodendrogliomas and mixed gliomas (gliomas with both oligodendroglial and astrocytic components) occur in low-grade (Grade II) and more malignant variants (Grade III).

As patient prognosis and therapeutic decisions are made in reliance on accurate pathological grading, consistency is a critical attribute. While for the most part reproducible, the present histological based system can result in substantial disagreement between neuropathologists with respect to both type and grade. Louis, D N et al., Am J. Pathol. 159: 779-86 (2001); Prayson R A et al., J. Neurol. Sci. 175: 33-9 (2000); Coons et al., Cancer 79:1381-93 (1997). Moreover, the precise method of grading changes over time. Because it is based on morphology [Burger, Brain Pathol. 12:257-9 (2002)], a biological, rather than molecular end state, the histological approach is limited in its ability to identify new potential compounds. It has been noted from a variety of treatment regimens, that clinical responses to histologically identical tumors can be highly varied. Mischel et al., supra.; Cloughesy, T F et al., Cancer 97: 2381-6 (2003). This underscores how histopathologic evaluation does not necessarily reveal the underlying molecular biology. As oncologists move to molecularly targeted therapies, identification of distinct molecularly defined subgroups becomes increasingly important to both treatment and diagnosis.

Microarray analysis has been identified as a tool that can provide unbiased, quantitative and reproducible tumor evaluation because it can simultaneously evaluate the expression thousands of individual genes. This approach as been applied to many different cancers including gliomas. Mischel, P. S. et al., Oncogene 22: 2361-73 (2003); Kim, S. et al., Mol. Cancer. Ther. 1:1229-36 (2002); Ljubimova et al., Cancer Res. 61: 5601-10 (2001); Nutt, C L et al., Cancer Res. 63: 1602-7 (2003); Rickman, D. S. et al., Cancer Res. 61: 6885-91 (2001); Sallinen, S. L. et al., Cancer Res. 60: 6617-22 (2000); Shai, R. et al., Oncogene 22: 4918-23 (2003). Unlike histological evaluation, microarray analysis can identify the underlying genetic variation in the tumors, enhancing tumor classification as well as patient prognostication. Microarray analysis of gliomas has resulted in a classification into more homogenous groups. Freije et al., Cancer Res. 64: 6503-6510 (2004). Moreover, it has also been found to be a superior indicator of survival than histological grading. Freije et al., supra.

Expression profiling of malignant gliomas has identified molecular subtypes as well as genes associated with tumor grade progression, and patient survival. While GBM and astrocytoma continue to be defined on the basis of histologic appearance, the finding that expression profiling predicts outcome better than histological features provides support for the hypothesis that neoplasms defined as astrocytoma and GBM on a morphologic basis may represent a mix of subtypes differing at the molecular level. Given the possibility that molecularly-distinct disease entities may exhibit different clinical responses to targeted anti-cancer agents, a greater understanding of the behavior of molecularly-defined subsets of tumors may aid in the development of more effective therapeutics.

Attempts to discover effective cellular targets for cancer therapy and diagnosis have resulted in the search for polypeptides that are specifically overexpressed in a particular type of cancer cell as compared to non-cancerous cell(s). The kinases that control signal transduction pathways, cell cycle and programmed cell death are critical to cell regulation. Overexpression or activating mutations of these critical kinases may disrupt cellular regulation and lead to tumor formation. Twenty percent of all known oncogenes are protein kinases. Identifying the appropriate signal transduction pathway and developing drugs to specifically inhibit these oncogenic kinases has been a major goal of cancer research for years. High throughput screening has led to identification of small molecules with different modes of inhibition such as; competition with the catalytic adenosine triphosphate binding site, inhibition of substrate binding, or modification of the substrate itself. Certain compounds are highly specific for a single kinase, while others can inhibit several kinases with similar binding structures (Busse et al., Semin. Oncol. 28: 47-55 (2001)). For example, the tyrosine kinase Bcr-Abl has been identified as a causative factor in chronic myeloid leukemia (CML). The small molecule imatinib mesylate (Gleevec-Novartis Pharmaceuticals Corp, East Hanover, N.J.) was recently approved for the treatment of CML, demonstrating that treatment of the kinase component of a signal transduction pathway is effective in the treatment of cancer (Griffin, J. Semin. Oncol. 28: 3-8 (2001)).

Amplifications in the gene EGFR, often accompanied by the activating mutation IGFRvIII, have been reported in 30-50% of human GBMs. Friedman et al., N. Engl. J. Med. 353: 1997-99 (2005); Nutt et al., Cancer of the Nervous System, 2d Ed., Ch. 59: 837-847 (2005). Alterations in other growth-factor induced signaling cascases include amplification and/or overexpression of PDGFRα, PDGFRβ, PDGF and c-Met receptor and have typically been described in gliomas with unamplified EGFR. Nutt et al., Cancer of the Nervous System, 2d Ed., Ch. 59: 837-847 (2005); Wullich et al., Anticancer Res. 14: 577-79 (1994). Mouse models provide compelling evidence for the ability of EGFR or PDGF expression in neural progenitors to cooperate with inactivation of p53 or INK4A/ARF to drive the formation of leasions that closely resemble the histopathology of human gliomas. Dai et al., Genes Dev. 15: 1913-25 (2001); Hesselager et al., Cancer Res. 63: 4305-09 (2003); Holland et al., Genes Dev. 12: 3644-49 (1998); Shih et al., Cancer Res. 64: 4783-89 (2004).

While many genomic alterations are associated with GBM, the most common loss-of-function mutation is with PTEN, which occurs with an estimate frequency of 70-90% in all GBMs. Nutt et al., supra. While largely absent in lower grade astrocytomomas, PTEN loss-of-function is frequent in both GBMs which arise de novo as well as those which evolve from lower grade lesions. Rasheed et al., Cancer Res. 57: 4187-90 (1997). These findings, along with the prognostic value of PTEN status in GBM cases [Phillips et al., Cancer Cell 9 (3): 157-173 (2006)], suggest the importance of the PI3K/Akt pathway in promoting features characteristic of highly aggressive glial malignancies, such as proliferation and/or angiogenesis. Recently identified genetic alterations in the catalytic and regulatory subunits of the PI3 kinase add further evidence to suggest the importance of PI3K/Akt signaling in promoting human GBMs. Mizoguchi et al., Brain Pathol. 14: 372-77 (2004); Broderick et al., Cancer Res. 64: 5048-50 (2004); Samuels et al., Science 304: 554 (2004). Furthermore, experimental evidence shows that PTEN loss increases the pool of self-renewing neural stem cells and induces loss of homeostatic control of proliferation [Groszer et al., Proc. Natl. Acad. Sci. USA 103: 111-116 (2006)], a phenomenon reminiscent of cell cycle dysregulation that occurs during gliomagenesis. Taken together, this growing body of evidence indicates that the PI3K/Akt signaling axis, engaged downstream of growth factors and their receptors, functions a “master regulator” during both neurogenesis and glioma formation. Thus, inhibiting PI3K/Akt signaling is a promising approach to treating glioma.

PIK3R3 (also known as p55^{PIK} (p55 γ) is the regulatory subunit of PI3-kinase (PI3K), and is associated with the IGF signaling pathway (Pons et al., Mol. Cell. Bio. 15: 4453-4465 (1995)). PIK3R3 was first cloned from the mouse by screening an adiopose cell cDNA library. The PIK3R3 polypeptide was found to be tyrosine phosphorylated on a novel motif during insulin stimulation (Pons et al., supra). The human PIK3R3 was cloned from a human fetal brain library by yeast-two hybrid interaction with the intracellular domain of Insulin-like growth factor receptor I (IGFRI) (Dey et al., Gene 209: 175-183 (1998). PIK3R3 was shown to interact with IGFRI in a kinase dependent manner, providing an alternative pathway for the activation of PI3K via IGFRI. Dey et al, supra. In development of the brain, PIK3R3 is highly expressed in the cerebellum, and co-localizes in Perkinje cells with IGF1R, the receptor for IGF2 (Trejo et al., J. Neurobio. 47: 39-50 (2001)). Furthermore, in cells stimulated with IGFI, PIK3R3 coimmunoprecipitates with IGFRI (Mothe et al., Mol. Endo. 11: 1911-1923 (1997)). Given the importance of PI3K signaling for promoting cellular phenotypes associated with highly aggressive glial cancers, it is important to determine if PIK3R3 plays a role in GBM and to find associated therapeutics and diagnostics.

Applicants identify herein a subset of human GBMs that overexpress IGF2, which are mutually exclusive of tumors overexpressing EGFR. We further show that IGF2 induces the association of PIK3R3 with IGFRI, as well the involvement of the IGF2-PIK3R3 signaling axis in promoting the growth of a subset of highly aggressive human GBM tumors. As a result, there still exists the need for therapeutics targeted to Akt/PIK3 activation originating from IGF2-PIK3R3 signaling, for the diagnosis and treatment of glioma.

### SUMMARY OF THE INVENTION

The present invention provides generally for a method of diagnosing, prognosing and treating glioma. More specifically, the invention provides for a method of using activation of IGF2-PIK3R3 as a surrogate marker for diagnosing the severity of glioma in tumors. In one sense, the invention provides for a method of treating glioma by antagonizing IGF2-PIK3R3 signaling. In another sense, the invention provides for a method of antagonizing Akt/PI3K signaling through antagonizing IGF2-PIK3R3 signaling in glioma cells.

In one embodiment, the invention is directed to a method for inhibiting the growth of a glioma tumor that expresses a PIK3R3 polypeptide, wherein the growth of said glioma tumor is at least in part dependent upon the growth potentiating effect(s) of a PIK3R3 polypeptide, wherein the method comprises contacting the a cell of the glioma tumor with an effective amount of a PIK3R3 antagonist. In a specific aspect, the glioma tumor does not overexpress an EGFR polypeptide. In another specific aspect, the glioma tumor overexpresses an IgF2 polypeptide. In yet another specific aspect, the PIK3R3 antagonist binds to nucleic acid encoding a PIK3R3 polypeptide, thereby antagonizing Akt/PIK3 signaling, which in turn, inhibits the growth of the glioma cell. In a further specific aspect, the nucleic acid is DNA. In a further specific aspect, the nucleic acid is RNA. In yet a further specific aspect, the growth of the glioma cell is completely inhibited. In yet a further specific aspect, the growth inhibition results in the death of the cell. In yet a further specific aspect, the PIK3R3 antagonist is a PIK3R3 RNAi. In yet a further specific aspect, the method further comprises contacting the glioma tumor, prior, after or simultaneously, with an effective amount of an Akt and/or IgF2 antagonist. In yet a further specific aspect, the Akt antagonist is an antagonist of the catalytic or regulatory domain of PIK3 kinase.

In another embodiment, the invention is directed to a method of treating a glioma tumor in a mammal, wherein said tumor expresses a PIK3R3 polypeptide, wherein the method comprises administering to the mammal a therapeutically effective amount of a PIK3R3 antagonist. In a specific aspect, the glioma tumor does not overexpress an EGFR polypeptide. In a specific aspect, the glioma tumor does not overexpress an EGFR polypeptide. In another specific aspect, the glioma tumor overexpresses an IgF2 polypeptide. In another specific aspect, the PIK3R3 antagonist binds to nucleic acid encoding a PIK3R3 polypeptide, in a manner so as to antagonize Akt/PIK3 signaling. In yet another specific aspect, the administration of PIK3R3 antagonist results in reduced growth, shrinkage in volume or death of the glioma tumor. In a further specific aspect, the nucleic acid is DNA. In a further specific aspect, the nucleic acid is RNA. In a further specific aspect, the PIK3R3 antagonist is a PIK3R3 RNAi. In yet a further specific aspect, the method further comprises administering, prior, after or simultaneously, with a therapeutically effective amount of an Akt and/or IgF2 antagonist. In a specific aspect, the Akt antagonist is an antagonist of the catalytic or regulatory domain of PIK3 kinase.

In another embodiment, the invention is directed to a composition useful for diagnosing or treating a glioma tumor in a mammal, wherein said tumor expresses a PIK3R3 polypeptide, wherein the composition comprises an effective amount of a PIK3R3 antagonist. In another embodiment, the invention is directed to the use of an effective amount of a PIK3R3 antagonist for the manufacture of a medicament for diagnosing or treating a glioma tumor in a mammal, wherein the tumor expresses a PIK3R3 polypeptide. In yet another embodiment, the invention is directed to the use of an effective amount of a PIK3R3 antagonist for diagnosing or treating a glioma tumor in a mammal, wherein the tumor expresses a PIK3R3 polypeptide. In a specific aspect, the glioma tumor does not overexpress an EGFR polypeptide. In another specific aspect, the glioma tumor overexpresses an IgF2 polypeptide. In another specific aspect, the PIK3R3 antagonist binds to nucleic acid encoding a PIK3R3 polypeptide, in a manner so as to antagonize Akt/PIK3 signaling. In yet another specific aspect, the administration of PIK3R3 antagonist results in reduced growth, shrinkage in volume or death of the glioma tumor. In a further specific aspect, the nucleic acid is DNA. In a further specific aspect, the nucleic acid is RNA. In a further specific aspect, the PIK3R3 antagonist is a PIK3R3 RNAi. In yet a further specific aspect, the composition further comprises a therapeutically effective amount of an Akt and/or IgF2 antagonist. In a specific aspect, the Akt antagonist is an antagonist of the catalytic or regulatory domain of PIK3 kinase.

In yet another embodiment, the present invention is directed to a method of diagnosing the presence of a glioma tumor in a mammal, wherein the method comprises comparing the level of expression of a PIK3R3 polypeptide or nucleic acid encoding a PIK3R3 polypeptide (a) in a test sample of glioma tissue obtained from said mammal suspected of being cancerous, and (b) in a control sample of known normal cells of the same tissue origin, wherein a higher level of expression of the PIK3R3 polypeptide or nucleic acid encoding PIK3R3 polypeptide in the test sample, as compared to the control sample, is indicative of the presence of a glioma tumor in the mammal from which the test sample was obtained. In a specific aspect, the method of comparing the level of PIK3R3 expression is measured by a PIK3R3 nucleic acid, anti-PIK3R3 antibody, PIK3R3-binding antibody fragment, or PIK3R3 binding oligopeptide, PIK3R3 small molecule, antisense oligonucleotide or PIK3R3 RNAi.

In yet a further embodiment, the present invention is directed to a method of diagnosing the severity of a glioma tumor in a mammal, wherein the method comprises: (a) contacting a test sample comprising cells from said glioma tumor or extracts of DNA, RNA, protein or other gene product(s) obtained from the mammal with a reagent that binds to the PIK3R3 polypeptide or nucleic acid encoding PIK3R3 polypeptide in the sample, (b) measuring the amount of complex formation between the reagent with the PIK3R3-encoding nucleic acid or PIK3R3 polypeptide in the test sample, wherein the formation of a high level of complex, relative to the level in a known healthy sample of similar tissue origin, is indicative of an aggressive tumor. In a specific aspect, the PIK3R3 nucleic acid is DNA. In another specific aspect, the PIK3R3 nucleic acid is RNA. In yet another specific aspect, the method further comprises determining if the glioma tumor does not overexpress an EGFR polypeptide. In yet another specific aspect, the method further comprises determining if the glioma tumor overexpresses an IgF2 polypeptide. In a further specific aspect, the reagent(s) are/is detectably labeled, attached to a solid support, or the like useful for qualitatively and/or quantitatively determining the location and/or amount of binding or complex formation. In yet another specific aspect, the reagent is an anti-PIK3R3 antibody, PIK3R3-binding antibody fragment, or PIK3R3 binding oligopeptide, PIK3R3 small molecule, PIK3R3 nucleic acid, PIK3R3 RNAi or antisense oligonucleotide. In a further specific aspect, the anti-PIK3R3 antibody may be a monoclonal antibody, antigen-binding antibody fragment, chimeric antibody, humanized antibody or single-chain antibody.

In yet a further embodiment, the present invention is directed to a method of screening for PIK3R3 antagonists, comprising (a) contacting a test sample of PIK3R3 expressing glioma cells with a test compound and (b) comparing the expression of PIK3R3 in the contacted cells with control glioma cells that have not been contacted; wherein a lowered expression in the contacted cells is indicative of a PIK3R3 antagonist, and a therapeutic for the treatment of glioma tumors that are not overexpressing EGFR.

In yet a further embodiment, the invention is directed to a composition comprising a pharmaceutically-acceptable carrier, excipient or stabilizer and therapeutically effective amounts of (i) a PIK3R3 antagonist in combination with (ii) an IGF2 antagonist, with optionally (iii) an Akt antagonist.

In yet a further embodiment, the invention is directed to an article of manufacture comprising a container and a PIK3R3 antagonist, optionally further containing an Akt and/or IgF2 antagonist contained within the container, and instructions for use in the therapy, diagnosis and/or prognosis of glioma. The instruction may further comprise a label affixed to the container, or a package insert included with the container. In a specific aspect, the article of manufacture further comprises an IGF2 antagonist and optionally an Akt antagonist.

Further embodiments of the present invention will be evident to the skilled artisan upon a reading of the present specification.

### BRIEF DESCRIPTION OF THE DRAWINGS

FIGS. 1A-F. FIG. 1A is a heat map displaying microarray data for EGFR (upper panel) and IGF2 (lower panel) in a set of GBMs. Z score-normalized intensity values are depicted for Affymetrix® probes to EGFR or IGF2 as mapped to chromosomes 7p and 11p, respectively. FIG. 1B represents Affymetrix® intensity values for EGFR and IGF2 presented as a stacked bar graph for normal brain and all primary tumors. FIG. 1C is a Scatter plot for IGF2 and EGFR intensity values in grade III gliomas (filled circles) and GBMs (open circles) demonstrates no overlap between EGFR-OE and IGF2-OE cases. The dashed lines correspond to cut-off values for IGF2-OE (open) and EGFR-OE (dark). FIG. 1D is a graph of normalized mRNA levels (abundance relative to that of Rab14) for EGFR and IGF2 measured by Taqman in 12 selected cases. FIG. 1E is a comparison of EGFR CGH ratios vs. expression values shows strong correlation between amplification and overexpression. FIG. 1F shows IGF2 CGH ratios vs expression, shows no apparent genomic gains. In conclusion, FIGS. 1A-F show that EGFR-OE and IGF2-OE are non-overlapping across human GBM samples.

FIGS. 2A-J. FIGS. 2A & 2C. Phosphor-imager scans of TMA hybridized for IGF2 mRNA (A) or sense strand control probe (C) are depicted. FIGS. 2B & 2D are darkfield microphotographs of the tissue core indicated by grey boxes in (A) and (C), Bar=1 mm. In FIGS. 2A-D, IGF2 mRNA was detected by in situ hybridization using a ³³P-labeled IGF2 riboprobe. FIGS. 2E-G are tissue sections from an EGFR-positive case, showing IHC for EGFR (E), Ki-67 (F), and p-Akt (G). FIGS. 2H-J. Example of an IGF2-positive sample, showing darkfield micrograph of ISH for IGF2 (H), and IHC for Ki-67 (I) and p-Akt (J). Bar=100 μm. In conclusion, FIGS. 2A-J show that IGF2 positive tumors are highly proliferative and p-Akt positive.

FIGS. 3A-D. FIG. 3A is a G63 cell line (left panels), grown under control neurosphere conditions (top), in the presence of 20 ng/ml EGF (middle) or with 20 ng/ml IGF2 (bottom). Right panels show similar results obtained with neurospheres derived from acutely-dissociated GBM tissue. FIG. 3B is a proliferation assay of cells from neurospheres formed in the presence of IGF2 shows that IGF2 and EGF are equivalent mitogens for these cells. FIG. 3C depict EGF-derived neurospheres also respond similarly to either growth factor. FIG. 3D shows that IGF1R blocking antibody (α-IR3, 10 μg/ml) partially inhibits IGF2-induced cell proliferation (inhibition is significant for all shown concentrations—p<0.03). Figures B-D show representative results of three experiments/cell lines, with data presented as mean +/−S.D. In conclusion, FIGS. 3A-D show that IGF2 can substitute for EGF to support tumor-derived neurosphere growth.

FIGS. 4A-D. FIG. 4A is a stacked bar graph showing relative intensity values for both IGF2 and EGFR in 36 samples, selected to represent three molecular subtypes of high grade glioma: PN (proneural), Prolif (proliferative) and Mes (mesenchymal) as indicated. Values plotted represent intensity values of EGFR or IGF2 for each tumor normalized to the mean intensity of the corresponding gene across all cases. FIG. 4B shows that overexpression of PIK3R3 is found in cases with PIK3R3 genomic gains. Figures C & D show that the Affymetrix intensity values for PCNA, TOP2A, CDK2 and SMC4L1 are significantly elevated in cases with PIK3R3 genomic gains compared to those with no gain (C, p<0.001, t test, all comparisons) as well as in IGF2-OE cases compared to IGF2-non-overexpressing samples (D, p<0.05, t test, all comparisons). The data presented in FIGS. 4B-D are presented as mean +/−SEM. In conclusion, FIGS. 4A-D confirm that IGF2 and PIK3R3 are overexpressed in proliferative GBMs.

FIGS. 5A-C. FIGS. 5A-C are western blots of PIK3R3 immunoprecipitates of G96 cells following IGF2 stimulation. G96 cells were stimulated with IGF2 (20 ng/ml, 30 min), EGF (10 ng/ml, 30 min) or unstimulated. Blots were probed with antibodies as follows: A. (Upper) Antibody to IGF1R kinase domain; (Lower) Antibody to PIK3R3. B. (Upper) Antibody to phospho-IGF1R (PY1158/Y1621/Y1163); (Lower) Antibody to PIK3R3. C. (Upper) Antibody to phospho-Tyrosine; (Lower) Antibody to PIK3R3. In conclusion, FIGS. 5A-C show that IGF2 induces association between phospho-IGF1R and PIK3R3 in glioma cells.

FIGS. 6A-G. FIGS. 6A and B are Western blot analyses showing PIK3R3 knockdown in G96 (A) and G63 (B), respectively, resulting in decreased protein levels in the stable cell lines compared to control shRNA-treated cells. The shRNA2 construct (denoted by an asterisk) was chosen for growth studies of the G63 line. Figures C & D are representative neurosphere assays for PIK3R3 knock-down and control cells, and demonstrate that PIK3R3KD inhibits sphere formation and/or growth induced by either EGF or IGF2 in both G96 and G63 cell lines. Figures E & F represent the results of viability assays of dissociated spheres 14 days following initial plating, and show a decrease in the number of PIK3R3KD cells compared to control. The result is significant for all growth factor conditions, but most apparent under conditions of IGF2 stimulation. The significance of the effect of PIK3R3 knockdown for IGF2-stimulated spheres is p<0.005 for G96 and p<0.001 for G63; for EGF-grown neurospheres values are p<0.005 for G96 and <0.05 for G63; and for spheres exposed to neither IGF2 or EGF values are p<0.05 for G63 and P<0.01 for G96. Data are presented as mean +/−SD. FIG. 6G shows that in the absence of growth factor stimulation, p-Akt levels are equivalent in control and knockdown cells (left panels). Under IGF2 (20 ng/ml) stimulation, p-Akt levels in G96PIK3R3KD cells are visibly decreased compared to G96 control cells. In conclusion, FIGS. 6A-G show that PIK3R3 “knock down” inhibits IGF2-induced growth of glioma-derived neurospheres.

FIGS. 7A-B. FIGS. 7A-B shows a native sequence human PIK3R3 nucleic acid sequence (SEQ ID NO: 1), also known as Ref Seq: NM_{—}003629 or GenBank Accession: BC021622. The start and stop codons are shown in underlined, bold font in the figures.

FIG. 8 shows a native sequence PIK3R3 polypeptide (SEQ ID NO: 2) comprising the full-length amino acid encoded from the nucleic acid sequence shown in FIGS. 7A-B.

### DETAILED DESCRIPTION OF THE PREFERRED EMBODIMENTS

### I. Definitions

The terms “PIK3R3 polypeptide” and “PIK3R3” as used herein refers to specific polypeptide sequences as described herein. All disclosures in this specification which refer to the “PIK3R3 polypeptide” refer to each of the polypeptides individually as well as jointly. For example, descriptions of the preparation of, purification of, derivation of, formation of antibodies to or against, formation of PIK3R3 RNAi to or against, formation of PIK3R3 binding small molecules to or against, administration of, compositions containing, treatment of a disease with, etc., pertain to each polypeptide of the invention individually. The term “PIK3R3 polypeptide” also includes variants of the PIK3R3 polypeptides disclosed herein. The term “PIK3R3 nucleic acid” refers to nucleic acid (e.g., DNA, RNA etc.) that encodes a PIK3R3 polypeptide or fragment thereof.

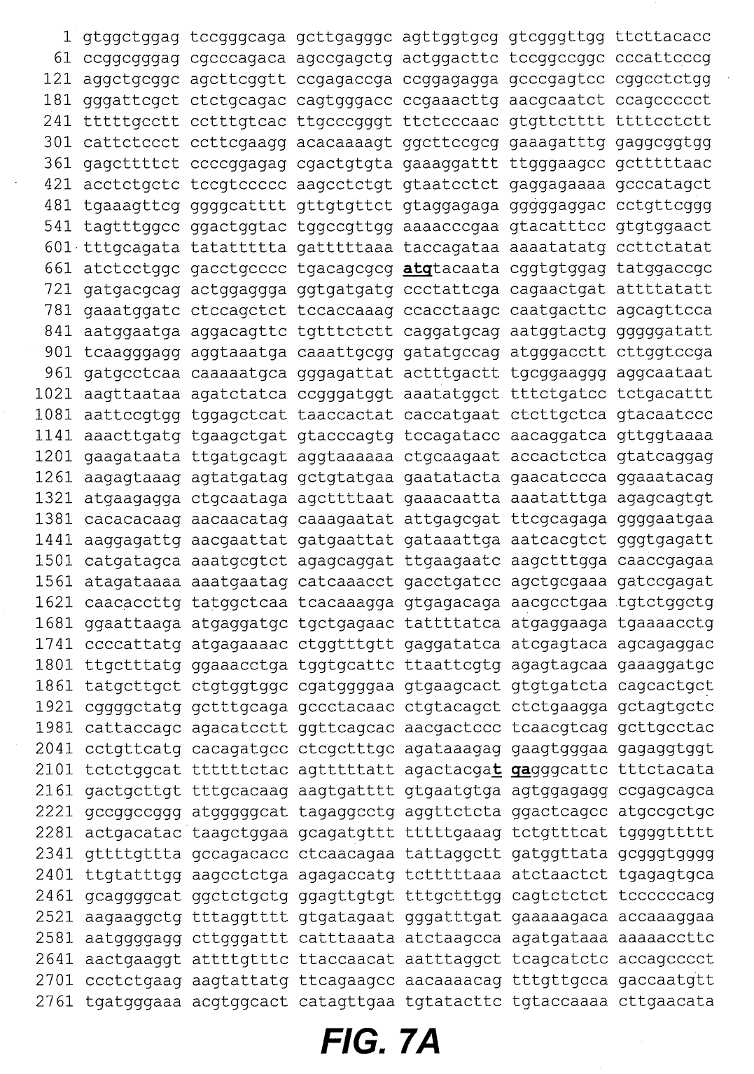

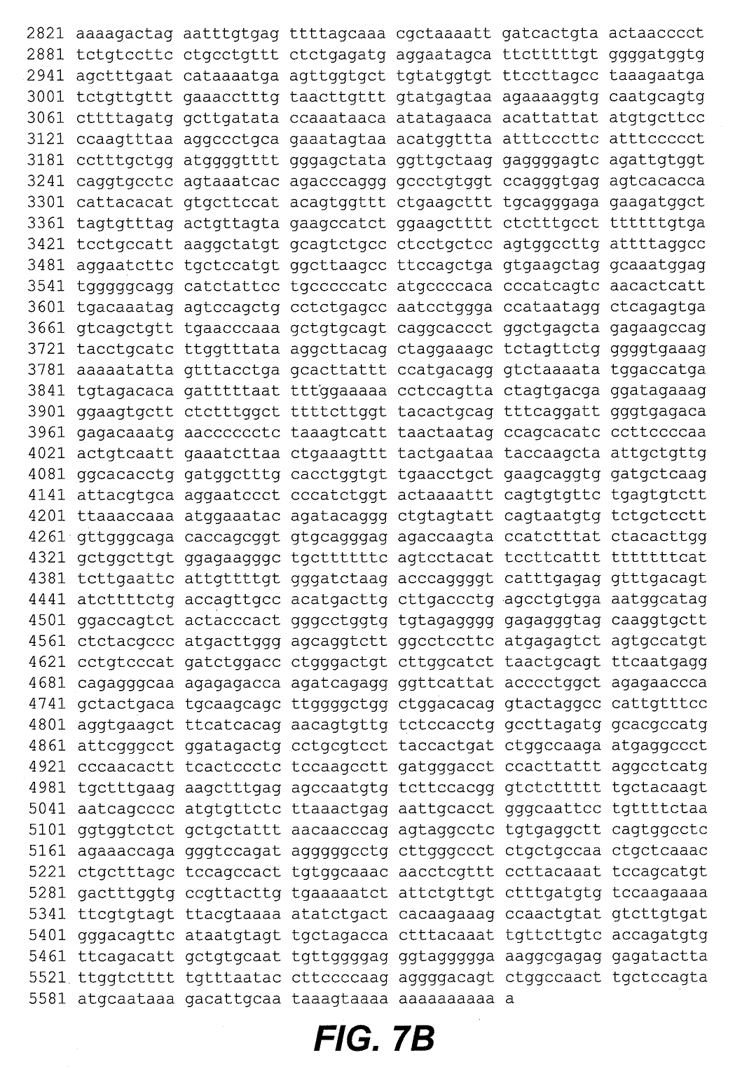

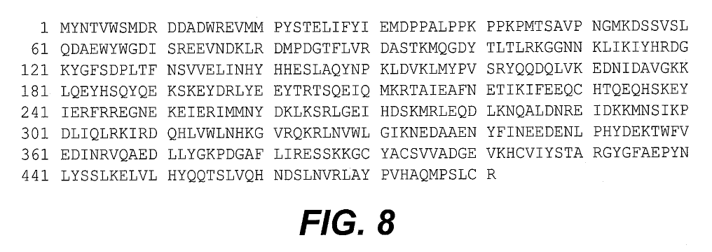

A “native sequence PIK3R3 polypeptide” comprises a polypeptide having the same amino acid sequence as the corresponding PIK3R3 polypeptide derived from nature. Such native sequence PIK3R3 polypeptides can be isolated from nature or can be produced by recombinant or synthetic means. The term “native sequence PIK3R3 polypeptide” specifically encompasses naturally-occurring truncated forms of the specific PIK3R3 polypeptide, naturally-occurring variant forms (e.g., alternatively spliced forms) and naturally-occurring allelic variants of the polypeptide, such as those encoded by a PIK3R3 polynucleotide sequence. In a specific embodiment, a native sequence PIK3R3 polypeptide is the mature or full-length native sequence polypeptides comprising the full-length amino acid sequence shown in FIG. 8. In another specific aspect, the native sequence PIK3R3 polypeptide sequence is encoded by the PIK3R3 polynucleotide sequence shown in FIGS. 7A-B.

A “PIK3R3 variants means a PIK3R3 polypeptide, preferably active forms thereof, as defined herein, having at least about 80% amino acid sequence identity with a full-length native sequence PIK3R3 polypeptide sequence, as disclosed herein, and variant forms of a full length native sequence PIK3R3 polypeptide such as those referenced herein. Such variant polypeptides include, for instance, polypeptides wherein one or more amino acid residues are added, or deleted, at the N- or C-terminus of the full-length native amino acid sequence. In a specific aspect, such variant polypeptides will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence PIK3R3 polypeptide sequence polypeptide, as disclosed herein, and variant forms of a full length native sequence PIK3R3 polypeptide polypeptide such as those disclosed herein. In a specific aspect, such variant polypeptides will vary at least about 1, 2, 3, 4, 5, 6, 7, 8, 9, 10, 20, 25, 30, 35, 40, 45, 50, 60, 70, 80, 90, 100, 125, 150, 200, 250, 300 or more amino acid residues in length from the corresponding native sequence polypeptide. Alternatively, such variant polypeptides will have no more than one conservative amino acid substitution as compared to the corresponding native polypeptide sequence, alternatively no more than 2, 3, 4, 5, 6, 7, 8, 9, or 10 conservative amino acid substitution as compared to the native polypeptide sequence.

“Percent (%) amino acid sequence identity” with respect to the PIK3R3 polypeptide sequences identified herein is defined as the percentage of amino acid residues in a candidate sequence that are identical with the amino acid residues in the specific PIK3R3 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PIK3R3”, wherein “PIK3R3” represents the amino acid sequence of a hypothetical PIK3R3 polypeptide of interest, “Comparison Protein” represents the amino acid sequence of a polypeptide against which the “PIK3R3” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

“PIK3R3 variant polynucleotide” or “PIK3R3 variant nucleic acid sequence” means a nucleic acid molecule which encodes a PIK3R3 polypeptide, preferably an active PIK3R3 polypeptide, as defined herein and which has at least about 80% nucleic acid sequence identity with a nucleotide acid sequence encoding a full-length native sequence PIK3R3 polypeptide sequence as disclosed herein, or any other fragment of a full-length PIK3R3 polypeptide sequence as disclosed herein (such as those encoded by a nucleic acid that represents only a portion of the complete coding sequence for a full-length PIK3R3 polypeptide). Ordinarily, a PIK3R3 variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence PIK3R3 polypeptide sequence as disclosed herein, or any other fragment of a full-length PIK3R3 polypeptide sequence as disclosed herein. Variants do not encompass the native nucleotide sequence. Alternatively, PIK3R3 variant polynucleotides are directed to isolated nucleic acid molecules which hybridize to (a) a nucleotide sequence encoding a PIK3R3 polypeptide having a full-length amino acid sequence as disclosed herein, or any other specifically defined fragment of a full-length PIK3R3 polypeptide amino acid sequence as disclosed herein, or (b) the complement of the nucleotide sequence of (a). Such PIK3R3 polynucleotide variants can be fragments of a full-length PIK3R3 polypeptide coding sequence, or the complement thereof, as disclosed herein, that may find use as, for example, hybridization probes useful as, for example, diagnostic probes, antisense oligonucleotide probes, or for encoding fragments of a full-length PIK3R3 polypeptide.

Ordinarily, PIK3R3 variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to PIK3R3-encoding nucleic acid sequences identified herein is defined as the percentage of nucleotides in a candidate sequence that are identical with the nucleotides in the PIK3R3 nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2. The ALIGN-2 sequence comparison computer program was authored by Genentech, Inc. and has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PIK3R3-DNA”, wherein “PIK3R3-DNA” represents a hypothetical PIK3R3-encoding nucleic acid sequence of interest, “Comparison DNA” represents the nucleotide sequence of a nucleic acid molecule against which the “PIK3R3-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, PIK3R3 variant polynucleotides are nucleic acid molecules that encode a PIK3R3 polypeptide and which are capable of hybridizing, preferably under stringent hybridization and wash conditions, to nucleotide sequences encoding a full-length PIK3R3 polypeptide as disclosed herein. PIK3R3 variant polypeptides may be those that are encoded by a PIK3R3 variant polynucleotide.

An “IgF2 polypeptide” includes native sequence and variants defined in a manner similarly to that for PIK3R3, above. Specifically encompassed within this definition is the polypeptide encoded, and variants thereof, by the nucleic acid defined by Ref. Seq.: NM_{—}000612, and which can bind to either IgF1R or IgF2R and/or stimulate Akt-PIK3 signaling in the absence of, or with insufficient expression of EGFR in a glioma cell.

“Isolated,” when used to describe the various PIK3R3 polypeptides disclosed herein, means a polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PIK3R3 polypeptide natural environment will not be present. Ordinarily, however, isolated polypeptide will be prepared by at least one purification step.

An “isolated” PIK3R3 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% FicoII/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 □g/ml), 0.1% SDS, and 10% dextran sulfate at 42° C., with washes at 42° C. in 0.2×SSC (sodium chloride/sodium citrate), followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

“Active” or “activity” for the purposes herein refers to form(s) of a PIK3R3 polypeptide which retain a biological and/or an immunological activity of native or naturally-occurring PIK3R3, wherein “biological” activity refers to a biological function (either inhibitory or stimulatory) caused by a native or naturally-occurring PIK3R3 other than the ability to induce the production of an antibody against an antigenic epitope possessed by a native or naturally-occurring PIK3R3 and an “immunological” activity refers to the ability to induce the production of an antibody against an antigenic epitope possessed by a native or naturally-occurring PIK3R3. An active PIK3R3 polypeptide, as used herein, is a PIK3R3 polypeptide that is expressed at an elevated level in a glioma tumor, relative to the expression of the polypeptide on similar tissue that is not afflicted with glioma. Alternatively, an active PIK3R3 polypeptide is a PIK3R3 polypeptide that is necessary for IgF2-induced PIK3-Akt signaling.

The term “antagonist” is used in the broadest sense, and includes any molecule that partially or fully blocks, inhibits or neutralizes a biological activity of the target polypeptide. A “PIK3R3 antagonist” includes any molecule that partially or fully blocks, inhibits or neutralizes the activity of a PIK3R3, so as attenuate Akt/PI3K signaling. Suitable examples of PIK3R3 antagonists include PIK3R3 RNAi molecules, PIK3R3 binding oligopeptides, PIK3R3 binding small molecules, PIK3R3 antisense oligonucleotides, anti-PIK3R3 antibodies and PIK3R3-binding fragments thereof, etc. Methods for identifying PIK3R3 antagonists may comprise contacting a PIK3R3 polypeptide, including a cell expressing it, with a candidate molecule and measuring a detectable change in one or more biological activities normally associated with the PIK3R3 polypeptide (e.g., Akt/PIK3 signaling).

An “Akt antagonist” is any molecule that partially or fully, blocks, inhibits, or neutralizes a biological activity of the Akt signaling pathway. Suitable Akt antagonists include antagonist antibodies or antigen binding fragments thereof, fragments or amino acid sequence variants of native components of the Akt signaling pathway, peptides, antisense oligonucleotides, small organic molecules, etc. Methods for identifying Akt antagonists may comprise contacting an Akt polypeptide with a candidate molecule and measuring a detectable change in one or more biological activities normally associated with the Akt polypeptide. Additional example Akt antagonists include: antagonists specifically directed to akt1, akt2 or akt3; antagonists directed at the catalytic or regulatory domain (including the interaction with each other) of PIK3 kinase such as PIK3CA, PIK3CB, PIK3CD, PIK3CG, PIK3R1, PIK3R2, PIK3R4; PDK1, FRAP (e.g., rapamycin), RPS6 KB1, SGK, EGFR (e.g., erlotinib TARCEVA®, IGFR. Alternatively, Akt antagonists include molecules that agonize, stimulate or restore activity of PTEN, INPP5D or INPPL1.

An “IgF2 antagonist” is any molecule that partially or fully blocks, inhibits or neutralizes a biological activity of an IgF2 polypeptide. Suitable IgF2 antagonists includes antibodies or antigen binding fragments thereof, fragments or amino acid fragments or amino acid sequence variants that bind to IgF2 in a manner that interferes with binding to IgF1R or IgF2R and/or IgF2-induced signaling in the Akt-PIK3 signaling axis. Methods for identifying IgF2 antagonists may comprise contacting an IgF2 polypeptide with a candidate molecule and measuring a detectable change in one or more biological activities normally associated with the IgF2 polypeptide.

“Treating” or “treatment” or “alleviation” refers to both therapeutic treatment and prophylactic or preventative measures, wherein the object is to prevent or slow down (lessen) the progression of glioma. “Prognosing” refer to the determination or prediction of the probable course and outcome of the glioma tumor. “Diagnosing” refer to the process of identifying or determining distinguishing characteristics of a glioma tumor.

Subjects in need of treatment, prognosis or diagnosis include those already with the disorder as well as those prone to have the disorder or those in whom the disorder is to be prevented. A subject or mammal is successfully “treated” for a PIK3R3 polypeptide-expressing glioma if, after receiving a therapeutic amount of s PIK3R3 antagonist according to the methods of the present invention, the patient shows observable and/or measurable reduction in or absence of one or more of the following: reduction in the number/absence of glioma cells; reduction in the tumor size; inhibition (i.e., slow to some extent and preferably stop) of glioma cell infiltration into peripheral organs including the spread of glioma into soft tissue and bone; inhibition (i.e., slow to some extent and preferably stop) of glioma metastasis; inhibition, to some extent, of tumor growth; and/or relief to some extent, one or more of the symptoms associated with the specific cancer; reduced morbidity and mortality, and improvement in quality of life issues. To the extent the PIK3R3 antagonist may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread areas outside of the immediate vicinity of the primary tumor. The invention described herein relates to the process of prognosing, diagnosing and/or treating involves the determination and evaluation of PIK3R3 amplication and expression.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a cancer refers to any animal classified as a mammal, including humans, domestic and farm animals, and zoo, sports, or pet animals, such as dogs, cats, cattle, horses, sheep, pigs, goats, rabbits, ferrets etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which a PIK3R3 antagonist of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of RNAi to a mammal. The components of the liposome are commonly arranged in a bilayer formation, similar to the lipid arrangement of biological membranes.

A “small” molecule or “small” organic molecule is defined herein to have a molecular weight below about 500 Daltons.

An “effective amount” of PIK3R3 antagonist is at least an amount sufficient to carry out a specifically stated purpose. An “effective amount” may be determined empirically and in a routine manner, in relation to the stated purpose by means of titration. For example, an effective amount of PIK3R3 antagonist which inhibits the growth of a glioma tumor is at least the minimal concentration required to achieve a reduction in the volume increase or progression of the tumor.

The term “therapeutically effective amount” refers to at least an amount of a PIK3R3 antagonist or other drug effective to “treat” a disease or disorder in a subject or mammal. In the case of glioma, the therapeutically effective amount of the drug may reduce the number of glioma cells; reduce the tumor size; inhibit (i.e., slow to some extent and preferably stop) glioma cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

A “growth inhibitory amount” of a PIK3R3 antagonist is an amount capable of inhibiting the growth of a cell, especially tumor, e.g., cancer cell, either in vitro or in vivo. Such a “growth inhibitory amount” for purposes of inhibiting neoplastic cell growth may be determined empirically and in a routine manner.

A “cytotoxic amount” of a PIK3R3 antagonist is an amount capable of causing the destruction of a cell, especially tumor, e.g., cancer cell, either in vitro or in vivo. Such a “cytotoxic amount” for purposes of inhibiting neoplastic cell growth may be determined empirically and in a routine manner.

An “interfering RNA” or RNAi is RNA of 10 to 50 nucleotides in length which reduces expression of a target gene, wherein portions of the strand are sufficiently complementary (e.g. having at least 80% identity to the target gene). The method of RNA interference refers to the target-specific suppression of gene expression (i.e., “gene silencing”), occurring at a post-transcriptional level (e.g., translation), and includes all posttranscriptional and transcriptional mechanisms of RNA mediated inhibition of gene expression, such as those described in P. D. Zamore, Science 296: 1265 (2002) and Hannan and Rossi, Nature 431: 371-378 (2004). As used herein, RNAi can be in the form of small interfering RNA (siRNA), short hairpin RNA (shRNA), and/or micro RNA (miRNA).

Such RNAi molecules are often a double stranded RNA complexes that may be expressed in the form of separate complementary or partially complementary RNA strands. Methods are well known in the art for designing double-stranded RNA complexes. For example, the design and synthesis of suitable shRNA and siRNA may be found in Sandy et al., BioTechniques 39: 215-224 (2005).

A “small interfering RNA” or siRNA is a double stranded RNA (dsRNA) duplex of 10 to 50 nucleotides in length which reduces expression of a target gene, wherein portions of the first strand is sufficiently complementary (e.g. having at least 80% identity to the target gene). siRNAs are designed specifically to avoid the anti-viral response characterized by elevated interferon synthesis, nonspecific protein synthesis inhibition and RNA degredation that often results in suicide or death of the cell associated with the use of RNAi in mammalian cells. Paddison et al., Proc Natl Acad Sci USA 99(3):1443-8. (2002).

The term “hairpin” refers to a looping RNA structure of 7-20 nucleotides.

A “short hairpin RNA” or shRNA is a single stranded RNA 10 to 50 nucleotides in length characterized by a hairpin turn which reduces expression of a target gene, wherein portions of the RNA strand are sufficiently complementary (e.g. having at least 80% identity to the target gene).

The term “stem-loop” refers to a pairing between two regions of the same molecule base-pair to form a double helix that ends in a short unpaired loop, giving a lollipop-shaped structure.

A “micro RNA” (previously known as stRNA) is a single stranded RNA of about 10 to 70 nucleotides in length that are initially transcribed as pre-miRNA characterized by a “stem-loop” structure, which are subsequently processed into mature miRNA after further processing through the RNA-induced silencing complex (RISC).

A “PIK3R3 interfering RNA” or “PIK3R3 RNAi” binds, preferably specifically, to a PIK3R3 nucleic acid and reduces its expression. This means the expression of the PIK3R3 molecule is lower with the RNAi present as compared to expression of the PIK3R3 molecule in a control where the RNAi is not present. PIK3R3 RNAi may be identified and synthesized using known methods (Shi Y., Trends in Genetics 19(1):9-12 (2003), WO2003056012, WO2003064621, WO2001/075164, WO2002/044321.

A “PIK3R3 oligopeptide” is an oligopeptide that binds, preferably specifically, to a PIK3R3 polypeptide, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

A “PIK3R3 small molecule antagonist” or “PI3KR3 small molecule” is an organic molecule other than an oligopeptide or antibody as defined herein that inhibits, preferably specifically, a PIK3R3 polypeptide and PIK3R3/IgF2 signaling pathway. Such PIK3R3/IGF2 signaling inhibition preferably inhibits the growth of glioma tumor cells expressing the PIK3R3 polypeptide. Such organic molecules may be identified and chemically synthesized using known methodology (see, e.g., PCT Publication Nos. WO2000/00823 and WO2000/39585). Such organic molecules are usually less than about 2000 daltons in size, alternatively less than about 1500, 750, 500, 250 or 200 daltons in size, are capable of binding, preferably specifically, to a GDM polypeptide as described herein, and may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

A PIK3R3 antagonist which “binds” a nucleic acid of interest, e.g. nucleic acid encoding a PIK3R3 polypeptide, is one that binds the target sequence with sufficient affinity such that the RNAi is useful as a diagnostic and/or therapeutic agent in targeting a cell or tissue expressing the antigen, and does not significantly cross-react with other target sequences. In such embodiments, the extent of binding of the PIK3R3 antagonist to a “non-target” sequence win be less than about 10% of the binding of the PIK3R3 antagonist to its particular target protein as determined by hybridization. With regard to the binding of RNAi, the term “specific binding” or “specifically binds to” or is “specific for” a particular nucleic acid means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

A PIK3R3 antagonist that “inhibits the growth of tumor cells expressing a PIK3R3 polypeptide” or a “growth inhibitory” PIK3R3 antagonist is one which results in measurable growth inhibition of cancer cells expressing or overexpressing the appropriate PIK3R3 polypeptide. Preferred growth inhibitory PIK3R3 antagonists inhibit growth of PIK3R3-expressing tumor cells by greater than 20%, preferably from about 20% to about 50%, and even more preferably, by greater than 50% (e.g., from about 50% to about 100%) as compared to the appropriate control, the control typically being tumor cells not treated with the oligopeptide, RNAi or other small molecule being tested. Growth inhibition of tumor cells in vivo can be determined in various ways such as is described in the Experimental Examples section below.

A PIK3R3 antagonist that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one that overexpresses a PIK3R3 polypeptide. Preferably the cell is a glioma tumor. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the PIK3R3 antagonist induces apoptosis resulting in about 2 to 50 fold, preferably about 5 to 50 fold, and most preferably about 10 to 50 fold, induction of annexin binding relative to untreated cell in an annexin binding assay.

A PIK3R3 antagonist that “induces cell death” is one which causes a viable cell to become nonviable. The cell is one which expresses a PIK3R3 polypeptide, preferably a cell that overexpresses a PIK3R3 polypeptide as compared to a normal cell of the same tissue type. Preferably, the cell is a glioma cancer cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the oligopeptide, RNAi or other small molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing PIK3R3 antagonists are those which induce PI uptake in the PI uptake assay in BT474 cells.

The term “antibody” is used in the broadest sense and specifically covers, for example, single anti-PIK3R3 monoclonal antibodies (including agonist, antagonist, and neutralizing antibodies), anti-PIK3R3 antibody compositions with polyepitopic specificity, polyclonal antibodies, single chain anti-PIK3R3 antibodies, and fragments of anti-PIK3R3 antibodies (see below) as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

The term “glioma” refers to a tumor that arises from glial cells or their precursors of the brain or spinal cord. Gliomas are histologically defined based on whether they exhibit primarily astrocytic or oligodendroglial morphology, and are graded by cellularity, nuclear atypia, necrosis, mitotic figures, and microvascular proliferation—all features associated with biologically aggressive behavior. Astrocytomas are of two main types—high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord. Some of the more common low-grade astrocytomas are: Juvenile Pilocytic Astrocytoma (JPA), Fibrillary Astrocytoma Pleomorphic Xantroastrocytoma (PXA) and Desembryoplastic Neuroepithelial Tumor (DNET). The two most common high-grade astrocytomas are Anaplastic Astrocytoma (AA) and Glioblastoma Multiforme (GBM).

“Tumor”, as used herein, refers to all neoplastic cell growth and proliferation of cell originating from or in proximity to the glia, whether malignant or benign, and all pre-cancerous and cancerous cells and tissues.

A cell that “expresses” a given polypeptide is a cell which expresses a measurable amount of such polypeptide that is endogenous or transfected into such cell. A glioma that “expresses” a given polypeptide is a glioma comprising cells that express such a polypeptide. Such glioma cells expressing such polypeptides optionally produce sufficient levels of such polypeptide(s), such that an antagonist against such polypeptides (e.g., PIK3R3, IGF2) can bind to a component thereof, and as a result have a therapeutic effect. In one aspect, a “PIK3R3-expressing glioma” or “IGF2-expressing glioma” optionally expresses sufficient levels of the PIK3R3 gene or IGF2 gene, respectively, such that a PIK3R3 RNAi or IGF2 RNAi, respectively, can bind and thereby suppress the function of the expression product so as to have a therapeutic effect. A cancer that “overexpresses” a given polypeptide, [E.g., a (i) PIK3R3 polypeptide or (ii) IGF2 polypeptide, respectively], is one that has significantly higher levels of such polypeptide [E.g., a (i) PIK3R3 polypeptide or other PIK3R3 gene products thereof, or (ii) IGF2 polypeptide or other IGF2 gene products thereof, respectively], compared to a noncancerous cell of the same tissue type. Such overexpression may be caused by gene amplification or by increased transcription or translation. Polypeptide overexpression may be determined in a diagnostic or prognostic assay by evaluating increased levels of the polypeptide present in the cell (e.g., via an immunohistochemistry assay using antibodies prepared against an isolated form of such polypeptide, which may be prepared using recombinant DNA technology from an isolated nucleic acid encoding such polypeptide; FACS analysis, etc.). Alternatively, or additionally, one may measure levels of nucleic acid or mRNA in the cell that encodes for the desired polypeptide, e.g., via fluorescent in situ hybridization using a nucleic acid based probe corresponding to a nucleic acid encoding such polypeptide or the complement thereof; (FISH; see WO98/45479 published October, 1998), Southern blotting, Northern blotting, or polymerase chain reaction (PCR) techniques, such as real time quantitative PCR (qRT-PCR). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

The word “label” when used herein refers to a detectable compound or composition which is conjugated directly or indirectly to the antibody, oligopeptide or other small molecule so as to generate a “labeled” antibody, oligopeptide or other small molecule. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

An “anti-mitotic agent” includes a molecule that partially or fully blocks, inhibits or otherwise interferes with mitosis that occurs during cell division. Example of such agents includes: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel. auristatins, maytansinoids.

An “anti-angiogenic agent” is a molecule that partially or fully blocks, inhibits or otherwise neutralizes the process of angiogenesis or vaculature formation, especially that which is associated with is associated with a disease or disorder. Many angiogenesis antagonists have been identified and are known in the arts, including those listed by Brem, Cancer Control 6(5): 436-458 (1999). Generally, angiogenesis antagonist comprises a molecule targeting a specific angiogenic factor or an angiogenesis pathway. In certain aspects, the angiogenesis antagonist is a protein composition such as an antibody targeting an angiogenic factor. An example angiogenic factor is VEGF (also sometimes known as “VEGF-A”), a 165-amino acid vascular endothelial cell growth factor and related 121-, 189-, and 206-amino acid vascular endothelial cell growth factors, as described by Leung et al. Science, 246:1306 (1989), and Houck et al. Mol. Endocrin., 5:1806 (1991), together with the naturally occurring allelic and processed forms thereof. The term “VEGF” is also used to refer to truncated forms of the polypeptide comprising amino acids 8 to 109 or 1 to 109 of the 165-amino acid human vascular endothelial cell growth factor. Such truncated versions of native VEGF have binding affinity for the Flt-1 (VEGF-R1) and KDR (VEGF-R2) receptors comparable to native VEGF.

An example anti-angioenic factor is a neutralizing anti-VEGF antibody. An “anti-VEGF antibody” is an antibody that binds specifically to VEGF. Preferably, the anti-VEGF antibody of the invention can be used as a therapeutic agent in targeting and interfering with diseases or conditions wherein the VEGF activity is involved. Such anti-VEGF antibody will usually not bind to other VEGF homologues such as VEGF-B or VEGF-C, nor other growth factors such as PlGF, PDGF or bFGF. A preferred anti-VEGF antibody is a monoclonal antibody that binds to the same epitope as the monoclonal anti-VEGF antibody A4.6.1 produced by hybridoma ATCC HB 10709. More preferably the anti-VEGF antibody is a recombinant humanized anti-VEGF monoclonal antibody comprising mutated human IgG1 framework regions and antigen-binding complementarity determining regions from the murine anti-hVEGF monoclonal antibody A.4.6.1, and generated according to Presta et al. (1997) Cancer Res. 57:4593-4599 (1997), including but not limited to the antibody known as bevacizumab (BV; Avastin™).

Alternatively, an anti-angiogenic agent can be any small molecule capable of neutralizing, blocking, inhibiting, abrogating, reducing or interfering with VEGF activities including its binding to one or more VEGF receptors (e.g., VEGFR1 and VEGFR2).

A “growth inhibitory agent” when used herein refers to a compound or composition which inhibits growth of a cell, especially a PIK3R3-expressing cancer cell, either in vitro or in vivo. Thus, the growth inhibitory agent may be one which significantly reduces the percentage of PIK3R3-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxycycline” is a member of the tetracycline family of antibiotics. The full chemical name of doxcycline is 1-dimethylamino-2,4-a,5,7,12-pentahydroxy-11-methyl-4,6-dioxo-1,4-a,11,11a,12,12a-hexahydrotetracene-3-carboxamide. Doxycycline will bind the TetR and relieve the TetR inhibition of the TetO.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-□ and -□; mullerian-inhibiting substance; mouse gonadotropin-associated peptide; inhibin; activin; vascular endothelial growth factor; integrin; thrombopoietin (TPO); nerve growth factors such as NGF-□; platelet-growth factor; transforming growth factors (TGFs) such as TGF-□ and TGF-□; insulin-like growth factor-I and -II; erythropoietin (EPO); osteoinductive factors; interferons such as interferon -□, -□, and -□; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-□ or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

TABLE 1

| | | |
|---|---|---|
| PIK3R3 | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PIK3R3 polypeptide) = 5 divided by 15 = 33.3%

TABLE 2

| | | |
|---|---|---|
| PIK3R3 | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PIK3R3 polypeptide) = 5 divided by 10 = 50%

TABLE 3

| | | |
|---|---|---|
| PIK3R3-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PIK3R3-DNA nucleic acid sequence) = 6 divided by 14 = 42.9%

TABLE 4

| | | |
|---|---|---|
| PIK3R3-DNA | NNNNNNNNNNNN | (Length = 12 nucleotides) |
| Comparison | NNNNLLLVV | (Length = 9 nucleotides) |
| DNA | | |

% nucleic acid sequence identity = (the number of identically matching nucleotides between the two nucleic acid sequences as determined by ALIGN-2) divided by (the total number of nucleotides of the PIK3R3-DNA nucleic acid sequence) = 4 divided by 12 = 33.3%

### II. Diagnostic and Therapeutic Methods of the Invention

Aberrant signaling initiated by growth factors and their receptors cooperates with loss of certain tumor suppressors to initiate and sustain glioma development. EGFR amplification represents a hallmark for a subclass of human GBMs [Friedman et al., N. Engl. J. Med. 353: 811-22 (2005); Nutt et al., Cancer of the Nervous System, 2d Ed., Ch. 59: 837-847 (2005) and mouse modeling experiments have demonstrated that activating mutations of EGFR acting in concert with p16 loss can promote gliomagenesis. Holland et al., Genes Dev. 12: 3675-85 (1998) The current study identifies IGF2 OE as a novel molecular marker of a subgroup of high-grade gliomas that do not exhibit EGFR amplification. IGF2 has been previously implicated in several types of neoplastic growth. In humans, IGF2 has been implicated in the development of malignancies of the lung, prostate, and adrenal gland. Cui et al., Science 299: 1753-55 (2003); Giordano et al., Am. J. Pathol. 162: 521-31 (2003); Li et al., Cell Tissue Res. 291: 469-79 (1998); Pollak et al., Cancer Metastasis Rev. 17: 383-90 (1998). Loss of IGF2 imprinting is linked to increased risk for developing colorectal cancer and Wilms' tumors. Cui et al., supra, Vu et al., Cancer Res. 63: 1900-05 (2003) (24, 28) In mouse models, overexpression of IGF2 can lead to the development of lung tumors [Fults et al., Neurosurg. Focus 19, E7 (2005); Moorehead et al., Oncogene 22: 853-57 (2003)], and loss of IGF2 imprinting promotes development of intestinal tumors. Sakatani et al., Science 307: 1976-78 (2005). Further., within the central nervous system, IGF2 has been shown to play an essential role in the induction of medulloblastomas in genetically-engineered murine models. Hahn et al., J. Biol. Chem. 275: 28341-44 (2000); Hultbeg et al., Cancer 72: 3282-86 (1993). While previous findings reported loss of imprinting and overexpression (“OE”) of IGF2 in meningiomas [Hultberg et al., Cancer 72: 3282-86 (1993); Muller et al., Eur. J. Cancer 36: 651-55 (2000)], reports on IGF2 expression in glioma have not yielded a consistent picture. Sandberg et al., Neurosci. Lett 93: 114-9 (1988); Uyeno et al., Cancer Res. 56: 5356-59 (1996). Our finding of strong IGF2 OE in a discrete subpopulation of GBMs which lack EGFR amplification suggests that IGF2 may drive the development and growth of some GBMs.

In the current study, we observed robust OE of IGF2 in a subset of high-grade gliomas which lack EGFR amplification or OE. CGH analysis confirmed the presence of EGFR amplification in ¼ of the GBM cases we investigated, but did not reveal any evidence of genomic gains flanking the IGF2 locus. While IGF2 gene imprinting status has not been directly investigated in the current work, the extent and robustness of IGF2 OE suggest that loss of imprinting alone could not be responsible for the increase in IGF2 mRNA levels. Thus, at present, it is not clear what genetic or epigenetic events lead to the strong OE of IGF2 mRNA in some high-grade gliomas. Regardless of the mechanism responsible for robust IGF2 OE, both the higher incidence of this event in grade IV astrocytomas and the association with a highly proliferative phenotype suggest that IGF2 plays a key role in promoting development and growth of some high-grade gliomas.

Our data shows that IGF2-OE GBMs are highly proliferative, a hallmark of aggressive disease and that IGF2 supports the growth of GBM-derived neurospheres. Interestingly, another recent report shows that IGF2-positive medulloblastoma cells in situ are restricted to a subpopulation which display intense Ki-67 staining and that cultured medulloblastoma-derived cells as well as cerebellar neuronal precursors are growth stimulated by IGF2. Hartmann et al., Am. J. Pathol. 166: 1153-62 (2005). These findings suggest that IGF2 may serve as an effective mitogen to promote growth of both medulloblastomas and glioblastomas, two forms of central nervous system malignancies both hypothesized to arise from neural stem and/or precursor cells. Singh et al., Cancer Res. 63: 5821-28 (2003); Singh et al., Nature 432: 396-401 (2004).

Recent identification of brain tumor stem-like cells has provided new insights into parallels that exist between gliomagenesis and normal brain development. Singh et al., Oncogene 23: 7267-73 (2004). Two recent studies have used embryonic brain-derived neurospheres to demonstrate that loss of PTEN [Groszer et al., supra.;] or p53 [Meletis et al., Development 133: 363-39 (2006)] enhances renewal and expansion of neural stem cells and promotes their “escape” from homeostasis, mechanisms also believed to underlie tumor initiation and progression. Both neural stem cells and stem-like cells from brain tumors maintained as neurospheres under the influence of EGF are self-renewing and maintain the potential to differentiate along either neuronal or glial lineages. Galli et al., Cancer Res. 64: 7011-21 (2004); Sanai et al., N. Engl. J. Med. 353: 811-22 (2005); Ignatova et al., Glia 39: 193-206 (2002); Doetsch et al., Neuron 36: 1021-34 (2002). In the current study, we show for the first time that IGF2 can support the growth of neurospheres derived from GBMs to the same extent as EGF; furthermore we demonstrate that IGF2-induced effects are mediated, at least in part, through the IGF1R. The equivalence of the EGF and IGF2 is underscored by the finding that growth responses to the two factors are virtually identical for neurospheres initially isolated and expanded under the influence of either growth factor. Interestingly, while IGF2 itself has not been previously demonstrated to play a role in supporting expansion of neural stem cells, insulin or insulin-like growth factors are required for successful maintenance of neural stem cells in culture [Ignatova et al., supra.; Arsenijevic et al., J. Neurosci. 21: 7194-202 (2001) and IGF2 has been shown to induce proliferation of cerebellar neuron precursors. Brooker et al., J. Neurosci. Res. 59: 332-41 (2000). During early stages of embryogenesis, IGF2 is produced in neural crest derivatives and mesenchymal structures [Dupont et al., Birth Defcts Res. C. Embryo Today 69: 257-71 (2003)], while the insulin-like growth factor-IGF1R axis has been reported to play an important role in development of neuronal and glial cells. Sara et al., Prog. Brain Res. 73: 87-99 (1988); Feldman et al., Neurobiol. Dis. 4: 201-14 (1997). Signaling through the IGF1R has also been implicated in oncogenic transformation, and both small molecule inhibitors, and neutralizing antibodies are currently tested for their in vivo efficiency in targeting this kinase. Cohen et al., Clin. Cancer Res. 11: 2063-73 (2005); Garcia-Echeverria et al., Cancer Cell 5: 231-39 (2004); Mitsiades et al., Cancer Cell 5: 221-30 (2004); Wang et al., Mol. Cancer. Ther. 4: 1214-21 (2005).

Herein, we demonstrate by multiple methods in independent sample sets that overexpress EGFR and IGF2 are mutually exclusive in high-grade gliomas, suggesting that either alteration is capable of supporting tumor growth. Our study of primary and recurrent case pairs, albeit in a modest number of cases, is especially intriguing. Primary tumors arising without overexpression of either IGF2 or EGFR invariably gave rise to recurrent lesions also lacking OE of both elements, suggesting that some lesions arise and are sustained by mechanisms independent of either EGFR or IGF1R signaling. Primary tumors with EGFR or IGF2 OE most frequently were associated with recurrences showing OE of mRNA for the same element. One case, however, was striking in its complete switch from an initial EGFR-OE/IGF2-negative primary tumor to a IGF2-OE/EGFR-negative recurrence. These findings suggest that tumors arising under the influence of either EGFR or IGF2 require continued growth factor signaling to sustain tumor growth and that IGF2-induced signaling may substitute for EGFR signaling in driving tumor growth. Taken together, these data support the notion that IGF2 OE may represent an alternate pathway to EGFR amplification in the development and growth of GBMs. The well-established actions of EGF and IGF2 in activating the PI3K/Akt signaling cascade suggests a mechanism by which these factors may exert parallel actions in supporting growth of high-grade gliomas.

The PI3K-Akt pathway plays a crucial role in supporting growth of several malignancies. Chow et al., Canc. Lett. 241 (2): 184-196 (2006); Cully et al., Nat. Rev. Cancer 6: 184-192 (2006). PTEN, a negative regulator of this pathway, has been called both a “master regulator” of neural precursor development [Groszer et al., Science 294: 2186-89 (2001)], as well as a potent tumor suppressor for gliomas. Several studies have shown that loss of PTEN is an important negative prognostic factor for GBM patients (see Phillips et al., supra). Genetic alterations in various catalytic subunits of the PI3 Kinase (PIK3CA and PIK3CD) have been recently described in human glioblastomas [Mizoguchi et al., Brain Pathol. 14: 372-77 (2004); Broderick et al., Cancr Res. 64: 5048-50 (2004); Samuels et al., Science 304: 554 (2004)], supporting the role of the PI3K pathway as an integrator of multiple signals essential for tumorigenesis. Cully et al., Nat. Rev. Cancer 6: 184-192 (2006). In the current work, we show that PTEN loss (assessed by CGH) and activation of the PI3K-Akt axis (assessed by pAkt IHC) are frequent occurrences in both EGFR-OE and IGF2-OE tumors. Furthermore, we present evidence that implicates a specific PI3K subunit in mediating IGF2 signaling in gliomas.

The regulatory subunit PIK3R3, also known as p55^{PIK} (p55 γ), was isolated by expression library screening for proteins interacting with phosphorylated IRS-1, [Pons et al., Mol. Cell. Biol. 15: 4453-65 (1995)] and was found to interact with the IGF1R using a yeast two hybrid screening approach. Dey et al., Gene 209: 175-83 (1998). During development, PIK3R3 is highly expressed in the cerebellum, where IGF1R and PIK3R3 were found co-localized in Purkinje cells. Trejo et al., J. Neurobiol. 47: 39-50 (2001). During CGH analysis of our glioma samples, we identified a subgroup of proliferative tumors that exhibit genomic gains for PIK3R3 and observed that these gains were associated with increased expression of mRNA of this molecule. Given that both GBMs with IGF2 OE and those with gains of PIK3R3 manifest a proliferative phenotype, we sought to determine whether PIK3R3 may be involved in mediating the growth-promoting effects of IGF2 on GBM cells. Previous studies using CHO cells transfected to overexpress PIK3R3 have shown that PIK3R3 associates with IGF1R and PDGFR, upon growth factor stimulation. Here, we present evidence in a GBM cell line that IGF2 stimulation induces endogenous PIK3R3 to associate with phosphorylated IGF1R and to appear as part of a tyrosine-phosphorylated intracellular complex. In addition, using glioma-derived neurospheres, we show that induction of both Akt phosphorylation and growth-stimulation by IGF2 (and, to a lesser extent, EGF) is inhibited by stable knockdown of PIK3R3. Importantly, our knockdown experiments were performed in two cell lines and using different shRNA constructs, arguing in favor of the specificity of the observed effect. These results are unexpected as mRNA for other regulatory subunits of PI3K (i.e., p85α□ and p85β) are present in both cell lines examined (data not shown) and might have been anticipated to substitute for the action of PIK3R3. This finding further supports the hypothesis that growth-promoting effects of IGF2 in human GBMs may be mediated in large part by engagement of PIK3R3.

In summary, our results reveal that robust expression of IGF2 is a marker for a subset of high-grade gliomas lacking EGFR amplification and provide evidence that IGF2 signaling via engagement of PIK3R3 can support growth of GBM cells in vitro. These findings suggest that IGF2 OE may serve as alternate mechanism to EGFR amplification for driving the formation and growth of GBMs, and that antagonists of PIK3R3 and/IGF2 in glioma tumors lacking EGFR overexpression would be useful therapeutics for the treatment of glioma.

### III. Compositions and General Methods of the Invention

A. Anti-PIK3R3 Antibodies

In one embodiment, the present invention provides anti-PIK3R3 antibodies which may find use herein as diagnostic agents. Exemplary antibodies include polyclonal and monoclonal antibodies, and fragments thereof.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 □g or 5 □g of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

3. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

4. Labelled Antibodies.

The antibodies of the invention may be conjugated with any label moiety which can be covalently attached to the antibody through a reactive functional group (Singh et al (2002) Anal. Biochem. 304:147-15; Harlow E. and Lane, D. (1999) Using Antibodies: A Laboratory Manual, Cold Springs Harbor Laboratory Press, Cold Spring Harbor, N.Y.; Lundblad R. L. (1991) Chemical Reagents for Protein Modification, 2nd ed. CRC Press, Boca Raton, Fla.). The attached label may function to: (i) provide a detectable signal; (ii) interact with a second label to modify the detectable signal provided by the first or second label, e.g. to give FRET (fluorescence resonance energy transfer); (iii) stabilize interactions or increase affinity of binding, with antigen or ligand; (iv) affect mobility, e.g. electrophoretic mobility or cell-permeability, by charge, hydrophobicity, shape, or other physical parameters, or (v) provide a capture moiety, to modulate ligand affinity, antibody/antigen binding, or ionic complexation.

Labelled antibodies may be useful in diagnostic assays, e.g., for detecting expression of an antigen of interest in specific cells, tissues, or serum. For diagnostic applications, the antibody will typically be labeled with a detectable moiety. Numerous labels are available which can be generally grouped into the following categories:
(a) Radioisotopes (radionuclides), such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labelled antibodies are useful in receptor targeted imaging experiments. The antibody can be labeled with ligand reagents that bind, chelate or otherwise complex a radioisotope metal where the reagent is reactive with a reactive nucleophile of the antibody such as a cysteine thiol, a lysine amine, or serine, threonine or tyrosine hydroxyl, using the techniques described in Current Protocols in Immunology, Volumes 1 and 2, Coligen et al, Ed. Wiley-Interscience, New York, N.Y., Pubs. (1991). Chelating ligands which may complex a metal ion include DOTA, DOTP, DOTMA, DTPA and TETA (Macrocyclics, Dallas, Tex.). Radionuclides can be targeted via complexation with the antibody-drug conjugates of the invention (Wu et al (2005) Nature Biotechnology 23(9):1137-1146).

Metal-chelate complexes suitable as antibody labels for imaging experiments are disclosed: Hnatowich et al (1983) J. Immunol. Methods 65:147-157; Meares et al (1984) Anal. Biochem. 142:68-78; Mirzadeh et al (1990) Bioconjugate Chem. 1:59-65; Meares et al (1990) J. Cancer 1990, Suppl. 10:21-26; Izard et al (1992) Bioconjugate Chem. 3:346-350; Nikula et al (1995) Nucl. Med. Biol. 22:387-90; Camera et al (1993) Nucl. Med. Biol. 20:955-62; Kukis et al (1998) J. Nucl. Med. 39:2105-2110; Verel et al (2003) J. Nucl. Med. 44:1663-1670; Camera et al (1994) J. Nucl. Med. 21:640-646; Ruegg et al (1990) Cancer Res. 50:4221-4226; Verel et al (2003) J. Nucl. Med. 44:1663-1670; Lee et al (2001) Cancer Res. 61:447-44482; Mitchell, et al (2003) J. Nucl. Med. 44:1105-1112; Kobayashi et al (1999) Bioconjugate Chem. 10:103-111; Miederer et al (2004) J. Nucl. Med. 45:129-137; DeNardo et al (1998) Clinical Cancer Research 4:2483-90; Blend et al (2003) Cancer Biotherapy & Radiopharmaceuticals 18:355-363; Nikula et al (1999) J. Nucl. Med. 40:166-76; Kobayashi et al (1998) J. Nucl. Med. 39:829-36; Mardirossian et al (1993) Nucl. Med. Biol. 20:65-74; Roselli et al (1999) Cancer Biotherapy & Radiopharmaceuticals, 14:209-20.

(b) Fluorescent labels such as rare earth chelates (europium chelates), fluorescein types including FITC, 5-carboxyfluorescein, 6-carboxy fluorescein; rhodamine types including TAMRA; dansyl; Lissamine; cyanines; phycoerythrins; Texas Red; and analogs thereof. The fluorescent labels can be conjugated to antibodies using the techniques disclosed in Current Protocols in Immunology, supra, for example. Fluorescent dyes and fluorescent label reagents include those which are commercially available from Invitrogen/Molecular Probes (Eugene, Oreg.) and Pierce Biotechnology, Inc. (Rockford, Ill.).
(c) Various enzyme-substrate labels are available or disclosed (U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al (1981) “Methods for the Preparation of Enzyme-Antibody Conjugates for use in Enzyme Immunoassay”, in Methods in Enzym. (ed J. Langone & H. Van Vunakis), Academic Press, New York, 73:147-166.
Examples of enzyme-substrate combinations include, for example
(i) Horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB));
(ii) alkaline phosphatase (AP) with para-nitrophenyl phosphate as chromogenic substrate; and
(iii) β-D-galactosidase (β-D-Gal) with a chromogenic substrate (e.g., p-nitrophenyl-β-D-galactosidase) or fluorogenic substrate 4-methylumbelliferyl-β-D-galactosidase.
Numerous other enzyme-substrate combinations are available to those skilled in the art. For a general review, see U.S. Pat. No. 4,275,149 and U.S. Pat. No. 4,318,980.

A label may be indirectly conjugated with an antibody. For example, the antibody can be conjugated with biotin and any of the three broad categories of labels mentioned above can be conjugated with avidin or streptavidin, or vice versa. Biotin binds selectively to streptavidin and thus, the label can be conjugated with the antibody in this indirect manner. Alternatively, to achieve indirect conjugation of the label with the polypeptide variant, the polypeptide variant is conjugated with a small hapten (e.g., digoxin) and one of the different types of labels mentioned above is conjugated with an anti-hapten polypeptide variant (e.g., anti-digoxin antibody). Thus, indirect conjugation of the label with the polypeptide variant can be achieved (Hermanson, G. (1996) in Bioconjugate Techniques Academic Press, San Diego).

The polypeptide variant of the present invention may be employed in any known assay method, such as ELISA, competitive binding assays, direct and indirect sandwich assays, and immunoprecipitation assays (Zola, (1987) Monoclonal Antibodies: A Manual of Techniques, pp. 147-158, CRC Press, Inc.).

A detection label may be useful for localizing, visualizing, and quantitating a binding or recognition event. The labelled antibodies of the invention can detect cell-surface receptors. Another use for detectably labelled antibodies is a method of bead-based immunocapture comprising conjugating a bead with a fluorescent labelled antibody and detecting a fluorescence signal upon binding of a ligand. Similar binding detection methodologies utilize the surface plasmon resonance (SPR) effect to measure and detect antibody-antigen interactions.

Detection labels such as fluorescent dyes and chemiluminescent dyes (Briggs et al (1997) “Synthesis of Functionalised Fluorescent Dyes and Their Coupling to Amines and Amino Acids,” J. Chem. Soc., Perkin-Trans. 1:1051-1058) provide a detectable signal and are generally applicable for labelling antibodies, preferably with the following properties: (i) the labelled antibody should produce a very high signal with low background so that small quantities of antibodies can be sensitively detected in both cell-free and cell-based assays; and (ii) the labelled antibody should be photostable so that the fluorescent signal may be observed, monitored and recorded without significant photo bleaching. For applications involving cell surface binding of labelled antibody to membranes or cell surfaces, especially live cells, the labels preferably (iii) have good water-solubility to achieve effective conjugate concentration and detection sensitivity and (iv) are non-toxic to living cells so as not to disrupt the normal metabolic processes of the cells or cause premature cell death.

Direct quantification of cellular fluorescence intensity and enumeration of fluorescently labelled events, e.g. cell surface binding of peptide-dye conjugates may be conducted on an system (FMAT® 8100 HTS System, Applied Biosystems, Foster City, Calif.) that automates mix-and-read, non-radioactive assays with live cells or beads (Miraglia, “Homogeneous cell- and bead-based assays for high throughput screening using fluorometric microvolume assay technology”, (1999) J. of Biomolecular Screening 4:193-204). Uses of labelled antibodies also include cell surface receptor binding assays, immunocapture assays, fluorescence linked immunosorbent assays (FLISA), caspase-cleavage (Zheng, “Caspase-3 controls both cytoplasmic and nuclear events associated with Fas-mediated apoptosis in vivo”, (1998) Proc. Natl. Acad. Sci. USA 95:618-23; U.S. Pat. No. 6,372,907), apoptosis (Vermes, “A novel assay for apoptosis. Flow cytometric detection of phosphatidylserine expression on early apoptotic cells using fluorescein labelled Annexin V” (1995) J. Immunol. Methods 184:39-51) and cytotoxicity assays. Fluorometric microvolume assay technology can be used to identify the up or down regulation by a molecule that is targeted to the cell surface (Swartzman, “A homogeneous and multiplexed immunoassay for high-throughput screening using fluorometric microvolume assay technology”, (1999) Anal. Biochem. 271:143-51).

Labelled antibodies of the invention are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Chen et al (2004) Bioconjugate Chem. 15:41-49; (v) bioluminescence; (vi) fluorescence; and (vii) ultrasound. Immunoscintigraphy is an imaging procedure in which antibodies labeled with radioactive substances are administered to an animal or human patient and a picture is taken of sites in the body where the antibody localizes (U.S. Pat. No. 6,528,624). Imaging biomarkers may be objectively measured and evaluated as an indicator of normal biological processes, pathogenic processes, or pharmacological responses to a therapeutic intervention. Biomarkers may be of several types: Type 0 are natural history markers of a disease and correlate longitudinally with known clinical indices, e.g. MRI assessment of synovial inflammation in rheumatoid arthritis; Type I markers capture the effect of an intervention in accordance with a mechanism-of-action, even though the mechanism may not be associated with clinical outcome; Type II markers function as surrogate endpoints where the change in, or signal from, the biomarker predicts a clinical benefit to “validate” the targeted response, such as measured bone erosion in rheumatoid arthritis by CT. Imaging biomarkers thus can provide pharmacodynamic (PD) therapeutic information about: (i) expression of a target protein, (ii) binding of a therapeutic to the target protein, i.e. selectivity, and (iii) clearance and half-life pharmacokinetic data. Advantages of in vivo imaging biomarkers relative to lab-based biomarkers include: non-invasive treatment, quantifiable, whole body assessment, repetitive dosing and assessment, i.e. multiple time points, and potentially transferable effects from preclinical (small animal) to clinical (human) results. For some applications, bioimaging supplants or minimizes the number of animal experiments in preclinical studies.

Radionuclide imaging labels include radionuclides such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. The radionuclide metal ion can be complexed with a chelating linker such as DOTA. Linker reagents such as DOTA-maleimide (4-maleimidobutyramidobenzyl-DOTA) can be prepared by the reaction of aminobenzyl-DOTA with 4-maleimidobutyric acid (Fluka) activated with isopropylchloroformate (Aldrich), following the procedure of Axworthy et al (2000) Proc. Natl. Acad. Sci. USA 97(4):1802-1807). DOTA-maleimide reagents react with a free cysteine amino acid of the antibodies and provide a metal complexing ligand on the antibody (Lewis et al (1998) Bioconj. Chem. 9:72-86). Chelating linker labelling reagents such as DOTA-NHS (1,4,7,10-tetraazacyclododecane-1,4,7,10-tetraacetic acid mono (N-hydroxysuccinimide ester) are commercially available (Macrocyclics, Dallas, Tex.). Receptor target imaging with radionuclide labelled antibodies can provide a marker of pathway activation by detection and quantitation of progressive accumulation of antibodies in tumor tissue (Albert et al (1998) Bioorg. Med. Chem. Lett. 8:1207-1210). The conjugated radio-metals may remain intracellular following lysosomal degradation.

Peptide labelling methods are well known. See Haugland, 2003, Molecular Probes Handbook of Fluorescent Probes and Research Chemicals, Molecular Probes, Inc.; Brinkley, 1992, Bioconjugate Chem. 3:2; Garman, (1997) Non-Radioactive Labelling: A Practical Approach, Academic Press, London; Means (1990) Bioconjugate Chem. 1:2; Glazer et al (1975) Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGryter, Berlin and New York; and Wong (1991) Chemistry of Protein Conjugation and Cross-linking, CRC Press, Boca Raton, Fla.); De Leon-Rodriguez et al (2004) Chem. Eur. J. 10:1149-1155; Lewis et al (2001) Bioconjugate Chem. 12:320-324; Li et al (2002) Bioconjugate Chem. 13:110-115; Mier et al (2005) Bioconjugate Chem. 16:240-237.

Peptides and proteins labelled with two moieties, a fluorescent reporter and quencher in sufficient proximity undergo fluorescence resonance energy transfer (FRET). Reporter groups are typically fluorescent dyes that are excited by light at a certain wavelength and transfer energy to an acceptor, or quencher, group, with the appropriate Stokes shift for emission at maximal brightness. Fluorescent dyes include molecules with extended aromaticity, such as fluorescein and rhodamine, and their derivatives. The fluorescent reporter may be partially or significantly quenched by the quencher moiety in an intact peptide. Upon cleavage of the peptide by a peptidase or protease, a detectable increase in fluorescence may be measured (Knight, C. (1995) “Fluorimetric Assays of Proteolytic Enzymes”, Methods in Enzymology, Academic Press, 248:18-34).

The labelled antibodies of the invention may also be used as an affinity purification agent. In this process, the labelled antibody is immobilized on a solid phase such a Sephadex resin or filter paper, using methods well known in the art. The immobilized antibody is contacted with a sample containing the antigen to be purified, and thereafter the support is washed with a suitable solvent that will remove substantially all the material in the sample except the antigen to be purified, which is bound to the immobilized polypeptide variant. Finally, the support is washed with another suitable solvent, such as glycine buffer, pH 5.0, that will release the antigen from the polypeptide variant.

Labelling reagents typically bear reactive functionality which may react (i) directly with a reactive nucleophilic group of an antibody to form the labelled antibody, (ii) with a linker reagent to form a linker-label intermediate, or (iii) with a linker antibody to form the labelled antibody. Reactive functionality of labelling reagents include: maleimide, haloacetyl, iodoacetamide succinimidyl ester (e.g. NHS, N-hydroxysuccinimide), isothiocyanate, sulfonyl chloride, 2,6-dichlorotriazinyl, pentafluorophenyl ester, and phosphoramidite, although other functional groups can also be used.

An exemplary reactive functional group is N-hydroxysuccinimidyl ester (NHS) of a carboxyl group substituent of a detectable label, e.g. biotin or a fluorescent dye. The NHS ester of the label may be preformed, isolated, purified, and/or characterized, or it may be formed in situ and reacted with a nucleophilic group of an antibody. Typically, the carboxyl form of the label is activated by reacting with some combination of a carbodiimide reagent, e.g. dicyclohexylcarbodiimide, diisopropylcarbodiimide, or a uronium reagent, e.g. TSTU (O—(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (0-(7-azabenzotriazol-1-yl)-N,N,N′,N′-tetramethyluronium hexafluorophosphate), an activator, such as 1-hydroxybenzotriazole (HOBt), and N-hydroxysuccinimide to give the NHS ester of the label. In some cases, the label and the antibody may be coupled by in situ activation of the label and reaction with the antibody to form the label-antibody conjugate in one step. Other activating and coupling reagents include TBTU (2-(1H-benzotriazo-1-yl)-1-1,3,3-tetramethyluronium hexafluorophosphate), TFFH (N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and aryl sulfonyl halides, e.g. triisopropylbenzenesulfonyl chloride.

B. PIK3R3 Binding Oligopeptides

PIK3R3 binding oligopeptides of the present invention are oligopeptides that bind, preferably specifically, to a PIK3R3 polypeptide as described herein. PIK3R3 binding oligopeptides may be chemically synthesized using known oligopeptide synthesis methodology or may be prepared and purified using recombinant technology. PIK3R3 binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a PIK3R3 polypeptide as described herein. PIK3R3 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10:173) and T7 phage display systems (Smith, G. P. and Scott, J. K. (1993) Methods in Enzymology, 217, 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol. Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

PIK3R3 peptides may also be expressed through an inducible system. The current invention provides for pHUSH-ProEx, an inducible selectable vector system. pHUSH-ProEx can also be packaged into active viral particles. Utility to pHUSH-ProEx can be found by combining it with PIK3R3 oligopeptides of the invention or useful fragments of a PIK3R3 polypeptide, and expressing either PIK3R3 fragments or PIK3R3 oligopeptides in such a manner as to inhibit the effect that a PIK3R3 polypeptide or fragment thereof has on cell proliferation.

C. PIK3R3 Small Molecules

PIK3R3 small molecules are small molecules other than oligopeptides or antibodies as defined herein that bind, preferably specifically, to a PIK3R3 polypeptide as described herein. PIK3R3 binding small molecules may be identified and chemically synthesized using known methodology (see, e.g., PCT Publication Nos. WO00/00823 and WO00/39585). PIK3R3 binding small molecules are usually about 500 daltons in size, alternatively less than about 1500, 750, 500, 250 or 200 daltons in size, wherein such small molecules that are capable of binding, preferably specifically, to a PIK3R3 polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). PIK3R3 binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. Screening for PIK3R3 Binding Oligopeptides, PIK3R3 Small Molecules and PIK3R3 RNAi with the Desired Properties

Techniques for generating antibodies, RNAi and small molecules that bind to PIK3R3 polypeptides have been described above. One may further select antibodies, RNAi or other small molecules with certain biological characteristics, as desired.

The growth inhibitory effects of an RNAi or other small molecule of the invention may be assessed by methods known in the art, e.g., using cells which express a PIK3R3 polypeptide either endogenously or following transfection with the PIK3R3 gene. For example, appropriate tumor cell lines and PIK3R3-transfected cells may be treated with an PIK3R3 RNAi or other small molecule of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an PIK3R3 RNAi or PIK3R3 binding small molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. Preferably, the tumor cell is one that overexpresses a PIK3R3 polypeptide. Preferably, the PIK3R3 RNAi or PIK3R3 binding small molecule will inhibit cell proliferation of a PIK3R3-expressing tumor cell in vitro or in vivo by about 25-100% compared to the untreated tumor cell, more preferably, by about 30-100%, and even more preferably by about 50-100% or 70-100%.

To select for an PIK3R3 RNAi or PIK3R3 binding small molecule which induces cell death, loss of membrane integrity as indicated by, e.g., propidium iodide (PI), trypan blue or 7AAD uptake may be assessed relative to control. A PI uptake assay can be performed in the absence of complement and immune effector cells. PIK3R3 polypeptide-expressing tumor cells are incubated with medium alone or medium containing the appropriate PIK3R3 RNAi or PIK3R3 binding small molecule. The cells are incubated for approximately a 3 day time period. Following each treatment, cells are washed and aliquoted into 35 mm strainer-capped 12×75 tubes (1 ml per tube, 3 tubes per treatment group) for removal of cell clumps. Tubes then receive PI (100 □g/ml). Samples may be analyzed using a FACSCAN® flow cytometer and FACSCONVERT® CellQuest software (Becton Dickinson). Those PIK3R3 RNAi or PIK3R3 binding small molecules that induce statistically significant levels of cell death as determined by PI uptake may be selected as cell death-inducing PIK3R3 RNAi or PIK3R3 binding small molecules.

To screen for oligopeptides or other small molecules which bind to an epitope on a PIK3R3 polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test oligopeptide or other small molecule binds the same site or epitope as a known anti-PIK3R3 antibody.

E. Full-Length PIK3R3 Polypeptides

The present invention also provides newly identified and isolated nucleotide sequences encoding polypeptides referred to in the present application as PIK3R3 polypeptides. In particular, cDNAs (partial and full-length) encoding various PIK3R3 polypeptides have been identified and isolated, as disclosed in further detail in the Examples below.

As disclosed in the Examples below, various cDNA clones have been described. The predicted amino acid sequence can be determined from the nucleotide sequence using routine skill. For the PIK3R3 polypeptides and encoding nucleic acids described herein, in some cases, Applicants have identified what is believed to be the reading frame best identifiable with the sequence information available at the time.

F. PIK3R3 Polypeptide Variants

In addition to the full-length native sequence PIK3R3 polypeptides described herein, it is contemplated that PIK3R3 polypeptide variants can be prepared. PIK3R3 polypeptide variants can be prepared by introducing appropriate nucleotide changes into the encoding DNA, and/or by synthesis of the desired polypeptide. Those skilled in the art will appreciate that amino acid changes may alter post-translational processes of the PIK3R3 polypeptide, such as changing the number or position of glycosylation sites or altering the membrane anchoring characteristics.

Variations in the PIK3R3 polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence as compared with the native sequence polypeptide. Optionally the variation is by substitution of at least one amino acid with any other amino acid in one or more of the domains of the PIK3R3 polypeptide. Guidance in determining which amino acid residue may be inserted, substituted or deleted without adversely affecting the desired activity may be found by comparing the sequence of the PIK3R3 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PIK3R3 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PIK3R3 polypeptide.

PIK3R3 polypeptide fragments may be prepared by any of a number of conventional techniques. Desired peptide fragments may be chemically synthesized. An alternative approach involves generating polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PIK3R3 polypeptide fragments share at least one biological and/or immunological activity with the native PIK3R3 polypeptide disclosed herein.

In particular embodiments, conservative substitutions of interest are shown in Table 5 under the heading of preferred substitutions. If such substitutions result in a change in biological activity, then more substantial changes, denominated exemplary substitutions in Table 6, or as further described below in reference to amino acid classes, are introduced and the products screened.

TABLE 5

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PIK3R3 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PIK3R3 polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the PIK3R3 polypeptide also may be substituted, generally with serine, to improve the oxidative stability of the molecule and prevent aberrant crosslinking. Conversely, cysteine bond(s) may be added to the PIK3R3 polypeptide to improve its stability.

G. Preparation of PIK3R3 Polypeptides

The description below relates primarily to production of PIK3R3 polypeptides by culturing cells transformed or transfected with a vector containing PIK3R3 polypeptide-encoding nucleic acid. It is, of course, contemplated that alternative methods, which are well known in the art, may be employed to prepare PIK3R3 polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PIK3R3 polypeptide may be chemically synthesized separately and combined using chemical or enzymatic methods to produce the desired PIK3R3 polypeptide.

1. Isolation of DNA Encoding PIK3R3 Polypeptide

DNA encoding PIK3R3 polypeptide may be obtained from a cDNA library prepared from tissue believed to possess the PIK3R3 polypeptide mRNA and to express it at a detectable level. Accordingly, human PIK3R3 polypeptide DNA can be conveniently obtained from a cDNA library prepared from human tissue. The PIK3R3 polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding the PIK3R3 polypeptide is to use PCR methodology [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PIK3R3 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac) 169 degP ompT kan^{r}; E. coli W3110 strain 37D6, which has the complete genotype tonA ptr3 phoA E15 (argF-lac) 169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PIK3R3 polypeptide-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharonzyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PIK3R3 polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for PIK3R3 polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding the PIK3R3 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PIK3R3 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the polypeptide. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces □-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2□ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PIK3R3 polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PIK3R3 polypeptide-encoding nucleic acid sequence to direct mRNA synthesis. Promoters recognized by a variety of potential host cells are well known. Promoters suitable for use with prokaryotic hosts include the □-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PIK3R3 polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PIK3R3 polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PIK3R3 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, □-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PIK3R3 polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the PIK3R3 polypeptide.

Still other methods, vectors, and host cells suitable for adaptation to the synthesis of the PIK3R3 polypeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the PIK3R3 polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PIK3R3 polypeptide or against a synthetic peptide based on the DNA sequences provided herein or against exogenous sequence fused to PIK3R3 DNA and encoding a specific antibody epitope.

6. Purification of PIK3R3 Polypeptide

Cells employed in expression of PIK3R3 polypeptide can be disrupted by various physical or chemical means, such as freeze-thaw cycling, sonication, mechanical disruption, or cell lysing agents.

It may be desired to purify the PIK3R3 polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PIK3R3 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PIK3R3 polypeptide produced.

H. Pharmaceutical Formulations

Therapeutic formulations of the PIK3R3 binding oligopeptides, PIK3R3 RNAi, PIK3R3 binding small molecules and/or PIK3R3 polypeptides used in accordance with the present invention are prepared for storage by mixing the polypeptide, oligopeptide, RNAi or small molecule having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The formulations herein may also contain more than one active compound as necessary for the particular indication being treated, preferably those with complementary activities that do not adversely affect each other. For example, in addition to a PIK3R3 binding oligopeptide, PIK3R3 RNAi, or PIK3R3 binding small molecule, it may be desirable to include in the one formulation, an additional RNAi, e.g., a second PIK3R3 RNAi which binds a different area on the PIK3R3 nucleic acid, or to some other target such as a growth factor that affects the growth of the particular cancer. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody or polypeptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and □ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

I. Diagnosis and Treatment of PIK3R3s Diagnosis and Treatment with Anti-PIK3R3 Antibodies, PIK3R3 Binding Oligopeptides, PIK3R3 siRNA and PIK3R3 Binding Small Molecules

To determine PIK3R3 expression in the cancer, various diagnostic assays are available. In one embodiment, PIK3R3 polypeptide overexpression may be analyzed by immunohistochemistry (IHC). Parrafin embedded tissue sections from a tumor biopsy may be subjected to the IHC assay and accorded a PIK3R3 protein staining intensity criteria as follows:

Score 0—no staining is observed or membrane staining is observed in less than 10% of tumor cells.

Score 1+—a faint/barely perceptible staining is detected in more than 10% of the tumor cells. The cells are only stained in part of their membrane.

Score 2+—a weak to moderate complete staining is observed in more than 10% of the tumor cells.

Score 3+—a moderate to strong complete staining is observed in more than 10% of the tumor cells.

Those tumors with 0 or 1+ scores for PIK3R3 polypeptide expression may be characterized as not overexpressing PIK3R3, whereas those tumors with 2+ or 3+ scores may be characterized as overexpressing PIK3R3.

Alternatively, or additionally, FISH assays such as the INFORM® (sold by Ventana, Ariz.) or PATHVISION® (Vysis, Ill.) may be carried out on formalin-fixed, paraffin-embedded tumor tissue to determine the extent (if any) of PIK3R3 overexpression in the tumor.

PIK3R3 overexpression or amplification may be evaluated using an in vivo diagnostic assay, e.g., by administering a molecule (such as an antibody, oligopeptide or small molecule) which binds the molecule to be detected and is tagged with a detectable label (e.g., a radioactive isotope or a fluorescent label) and externally scanning the patient for localization of the label.

As described above, the anti-PIK3R3 antibodies, oligopeptides and small molecules of the invention have various non-therapeutic applications. The anti-PIK3R3 antibodies, oligopeptides and small molecules of the present invention can be useful for diagnosis and staging of PIK3R3 polypeptide-expressing cancers (e.g., in radioimaging). The antibodies, oligopeptides and small molecules are also useful for purification or immunoprecipitation of PIK3R3 polypeptide from cells, for detection and quantitation of PIK3R3 polypeptide in vitro, e.g., in an ELISA or a Western blot, to kill and eliminate PIK3R3-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. Anti-PIK3R3 antibody, oligopeptide, siRNA or small molecule therapy may be especially desirable in elderly patients who do not tolerate the toxicity and side effects of chemotherapy well and in metastatic disease where radiation therapy has limited usefulness. The tumor targeting anti-PIK3R3 antibodies, oligopeptides, siRNA and small molecules of the invention are useful to alleviate PIK3R3-expressing cancers upon initial diagnosis of the disease or during relapse. For therapeutic applications, the anti-PIK3R3 antibody, oligopeptide, siRNA or small molecule can be used alone, or in combination therapy with, e.g., hormones, antiangiogens, or radiolabelled compounds, or with surgery, cryotherapy, and/or radiotherapy. Anti-PIK3R3 antibody, oligopeptide or small molecule treatment can be administered in conjunction with other forms of conventional therapy, either consecutively with, pre- or post-conventional therapy. Chemotherapeutic drugs such as TAXOTERE® (docetaxel), TAXOL® (palictaxel), estramustine and mitoxantrone are used in treating cancer, in particular, in good risk patients. In the present method of the invention for treating or alleviating cancer, the cancer patient can be administered anti-PIK3R3 antibody, oligopeptide or small molecule in conduction with treatment with the one or more of the preceding chemotherapeutic agents. In particular, combination therapy with palictaxel and modified derivatives (see, e.g., EP0600517) is contemplated. The anti-PIK3R3 antibody, oligopeptide or small molecule will be administered with a therapeutically effective dose of the chemotherapeutic agent. In another embodiment, the anti-PIK3R3 antibody, oligopeptide, siRNA or small molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

In one particular embodiment, a conjugate comprising an anti-PIK3R3 antibody, oligopeptide, or small molecule conjugated with a cytotoxic agent is administered to the patient. Preferably, the immunoconjugate bound to the PIK3R3 protein is internalized by the cell, resulting in increased therapeutic efficacy of the immunoconjugate in killing the cancer cell to which it binds. In a preferred embodiment, the cytotoxic agent targets or interferes with the nucleic acid in the cancer cell. Examples of such cytotoxic agents are described above and include maytansinoids, calicheamicins, ribonucleases and DNA endonucleases.

The anti-PIK3R3 antibodies, oligopeptides, small molecules or toxin conjugates thereof are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody, oligopeptide or small molecule is preferred.

Other therapeutic regimens may be combined with the administration of the anti-PIK3R3 antibody, oligopeptide or small molecule. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-PIK3R3 antibody or antibodies, oligopeptides or small molecules, with administration of an antibody directed against another tumor antigen associated with the particular cancer.

In another embodiment, the therapeutic treatment methods of the present invention involves the combined administration of an anti-PIK3R3 antibody (or antibodies), oligopeptides or small molecules and one or more chemotherapeutic agents or growth inhibitory agents, including co-administration of cocktails of different chemotherapeutic agents. Chemotherapeutic agents include estramustine phosphate, prednimustine, cisplatin, 5-fluorouracil, melphalan, cyclophosphamide, hydroxyurea and hydroxyureataxanes (such as paclitaxel and doxetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

The antibody, oligopeptide or small molecule may be combined with an anti-hormonal compound; e.g., an anti-estrogen compound such as tamoxifen; an anti-progesterone such as onapristone (see, EP 616 812); or an anti-androgen such as flutamide, in dosages known for such molecules. Where the cancer to be treated is androgen independent cancer, the patient may previously have been subjected to anti-androgen therapy and, after the cancer becomes androgen independent, the anti-PIK3R3 antibody, oligopeptide or small molecule (and optionally other agents as described herein) may be administered to the patient.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post antibody, oligopeptide or small molecule therapy. Suitable dosages for any of the above co-administered agents are those presently used and may be lowered due to the combined action (synergy) of the agent and anti-PIK3R3 antibody, oligopeptide or small molecule.

For the prevention or treatment of disease, the dosage and mode of administration will be chosen by the physician according to known criteria. The appropriate dosage of antibody, oligopeptide or small molecule will depend on the type of disease to be treated, as defined above, the severity and course of the disease, whether the antibody, oligopeptide or small molecule is administered for preventive or therapeutic purposes, previous therapy, the patient's clinical history and response to the antibody, oligopeptide or small molecule, and the discretion of the attending physician. The antibody, oligopeptide or small molecule is suitably administered to the patient at one time or over a series of treatments. Preferably, the antibody, oligopeptide or small molecule is administered by intravenous infusion or by subcutaneous injections. Depending on the type and severity of the disease, about 1 □g/kg to about 50 mg/kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-PIK3R3 antibody. However, other dosage regimens may be useful. A typical daily dosage might range from about 1 □g/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding the antibody is encompassed by the expression “administering a therapeutically effective amount of an antibody”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

The anti-PIK3R3 antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, immunoconjugates, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody competes for binding or bind substantially to, the same epitope as the antibodies of the invention. Antibodies having the biological characteristics of the present anti-PIK3R3 antibodies of the invention are also contemplated, specifically including the in vivo tumor targeting and any cell proliferation inhibition or cytotoxic characteristics.

Methods of producing the above antibodies are described in detail herein.

The present anti-PIK3R3 antibodies, oligopeptides and small molecules are useful for treating a PIK3R3-expressing cancer or alleviating one or more symptoms of the cancer in a mammal. Such a cancer includes GBM, glioma, astrocytoma and anaplastic astrocytoma. The cancers encompass metastatic cancers of any of the preceding. The antibody, oligopeptide or small molecule is able to bind to at least a portion of the cancer cells that express PIK3R3 polypeptide in the mammal. In a preferred embodiment, the antibody, oligopeptide or small molecule is effective to destroy or kill PIK3R3-expressing tumor cells or inhibit the growth of such tumor cells, in vitro or in vivo, upon binding to PIK3R3 polypeptide on the cell. Such an antibody includes a naked anti-PIK3R3 antibody (not conjugated to any agent). Naked antibodies that have cytotoxic or cell growth inhibition properties can be further harnessed with a cytotoxic agent to render them even more potent in tumor cell destruction. Cytotoxic properties can be conferred to an anti-PIK3R3 antibody by, e.g., conjugating the antibody with a cytotoxic agent, to form an immunoconjugate as described herein. The cytotoxic agent or a growth inhibitory agent is preferably a small molecule. Toxins such as calicheamicin or a maytansinoid and analogs or derivatives thereof, are preferable.

The invention provides a composition comprising an anti-PIK3R3 antibody, oligopeptide, siRNA or small molecule of the invention, and a carrier. For the purposes of treating cancer, compositions can be administered to the patient in need of such treatment, wherein the composition can comprise one or more anti-PIK3R3 antibodies present as an immunoconjugate or as the naked antibody. In a further embodiment, the compositions can comprise these antibodies, oligopeptides or small molecules in combination with other therapeutic agents such as cytotoxic or growth inhibitory agents, including chemotherapeutic agents. The invention also provides formulations comprising an anti-PIK3R3 antibody, oligopeptide or small molecule of the invention, and a carrier. In one embodiment, the formulation is a therapeutic formulation comprising a pharmaceutically acceptable carrier.

Another aspect of the invention is isolated nucleic acids encoding the anti-PIK3R3 antibodies. Nucleic acids encoding both the H and L chains and especially the hypervariable region residues, chains that encode the native sequence antibody as well as variants, modifications and humanized versions of the antibody, are encompassed.

The invention also provides methods useful for treating a PIK3R3 polypeptide-expressing cancer or alleviating one or more symptoms of the cancer in a mammal, comprising administering a therapeutically effective amount of an anti-PIK3R3 antibody, oligopeptide or small molecule to the mammal. The antibody, oligopeptide or small molecule therapeutic compositions can be administered short term (acute) or chronic, or intermittent as directed by physician. Also provided are methods of inhibiting the growth of, and killing a PIK3R3 polypeptide-expressing cell.

The invention also provides kits and articles of manufacture comprising at least one anti-PIK3R3 antibody, oligopeptide, siRNA or small molecule. Kits containing anti-PIK3R3 antibodies, oligopeptides, siRNA or small molecules find use, e.g., for PIK3R3 cell killing assays, for purification or immunoprecipitation of PIK3R3 polypeptide from cells. For example, for isolation and purification of PIK3R3, the kit can contain an anti-PIK3R3 antibody, oligopeptide or small molecule coupled to beads (e.g., sepharose beads). Kits can be provided which contain the antibodies, oligopeptides or small molecules for detection and quantitation of PIK3R3 in vitro, e.g., in an ELISA or a Western blot. Such antibody, oligopeptide or small molecule useful for detection may be provided with a label such as a fluorescent or radiolabel.

J. Articles of Manufacture and Kits

Another embodiment of the invention is an article of manufacture containing materials useful for the treatment of anti-PIK3R3 expressing cancer. The article of manufacture comprises a container and a label or package insert on or associated with the container. Suitable containers include, for example, bottles, vials, syringes, etc. The containers may be formed from a variety of materials such as glass or plastic. The container holds a composition, which is effective for treating the cancer condition, and may have a sterile access port (for example the container may be an intravenous solution bag or a vial having a stopper pierceable by a hypodermic injection needle). At least one active agent in the composition is an anti-PIK3R3 antibody, oligopeptide or small molecule of the invention. The label or package insert indicates that the composition is used for treating cancer. The label or package insert will further comprise instructions for administering the antibody, oligopeptide or small molecule composition to the cancer patient. Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for PIK3R3-expressing cell killing assays, for purification or immunoprecipitation of PIK3R3 polypeptide from cells. For isolation and purification of PIK3R3 polypeptide, the kit can contain an anti-PIK3R3 antibody, oligopeptide, siRNA or small molecule coupled to beads (e.g., sepharose beads). Kits can be provided which contain the antibodies, oligopeptides or small molecules for detection and quantitation of PIK3R3 polypeptide in vitro, e.g., in an ELISA or a Western blot. As with the article of manufacture, the kit comprises a container and a label or package insert on or associated with the container. The container holds a composition comprising at least one anti-PIK3R3 antibody, oligopeptide or small molecule of the invention. Additional containers may be included that contain, e.g., diluents and buffers, control antibodies. The label or package insert may provide a description of the composition as well as instructions for the intended in vitro or diagnostic use.

K. Uses for PIK3R3 Polypeptides and PIK3R3-Polypeptide Encoding Nucleic Acids

Nucleotide sequences (or their complement) encoding PIK3R3 polypeptides have various applications in the art of molecular biology, including uses as hybridization probes, in chromosome and gene mapping and in the generation of anti-sense RNA, siRNA and DNA probes. PIK3R3-encoding nucleic acid will also be useful for the preparation of PIK3R3 polypeptides by the recombinant techniques described herein, wherein those PIK3R3 polypeptides may find use, for example, in the preparation of anti-PIK3R3 antibodies as described herein.

The full-length native sequence PIK3R3 gene, or portions thereof, may be used as hybridization probes for a cDNA library to isolate the full-length PIK3R3 cDNA or to isolate still other cDNAs (for instance, those encoding naturally-occurring variants of PIK3R3 or PIK3R3 from other species) which have a desired sequence identity to the native PIK3R3 sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence PIK3R3. By way of example, a screening method will comprise isolating the coding region of the PIK3R3 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the PIK3R3 gene of the present invention can be used to screen libraries of human cDNA, genomic DNA or mRNA to determine which members of such libraries the probe hybridizes to. Hybridization techniques are described in further detail in the Examples below. Any EST sequences disclosed in the present application may similarly be employed as probes, using the methods disclosed herein.

Other useful fragments of the PIK3R3-encoding nucleic acids include antisense or sense oligonucleotides comprising a singe-stranded nucleic acid sequence (either RNA or DNA) capable of binding to target PIK3R3 mRNA (sense) or PIK3R3 DNA (antisense) sequences. Antisense or sense oligonucleotides, according to the present invention, comprise a fragment of the coding region of PIK3R3 DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the present invention. The antisense oligonucleotides thus may be used to block expression of PIK3R3 proteins, wherein those PIK3R3 proteins may play a role in the induction of cancer in mammals. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

Alternatively, a double stranded RNA can be generated. Double stranded RNAs that are under 30 nucleotides in length will inhibit the expression of specific genes when introduced into a cell. This mechanism is known as RNA mediated interference (RNAi) and small (under 30 nucleotides) RNAs used as a reagent are known as siRNAs. PIK3R3 interfering RNAs may be identified and synthesized using known methods (Shi Y., Trends in Genetics 19(1):9-12 (2003), WO/2003056012 and WO2003064621). siRNAs are useful to reduce the amount of gene expression in conditions where a reduction in the expression of the target gene would alleviate the condition or disorder.

The probes may also be employed in PCR techniques to generate a pool of sequences for identification of closely related PIK3R3 coding sequences.

Nucleotide sequences encoding a PIK3R3 can also be used to construct hybridization probes for mapping the gene that encodes that PIK3R3 and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for PIK3R3 encode a protein which binds to another protein (example, where the PIK3R3 is a receptor), the PIK3R3 can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor PIK3R3 can be used to isolate correlative ligand(s). Screening assays can be designed to find lead compounds that mimic the biological activity of a native PIK3R3 or a receptor for PIK3R3. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode PIK3R3 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding PIK3R3 can be used to clone genomic DNA encoding PIK3R3 in accordance with established techniques and the genomic sequences used to generate transgenic animals that contain cells which express DNA encoding PIK3R3. Methods for generating transgenic animals, particularly animals such as mice or rats, have become conventional in the art and are described, for example, in U.S. Pat. Nos. 4,736,866 and 4,870,009. Typically, particular cells would be targeted for PIK3R3 transgene incorporation with tissue-specific enhancers. Transgenic animals that include a copy of a transgene encoding PIK3R3 introduced into the germ line of the animal at an embryonic stage can be used to examine the effect of increased expression of DNA encoding PIK3R3. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of PIK3R3 can be used to construct a PIK3R3 “knock out” animal which has a defective or altered gene encoding PIK3R3 as a result of homologous recombination between the endogenous gene encoding PIK3R3 and altered genomic DNA encoding PIK3R3 introduced into an embryonic stem cell of the animal. For example, cDNA encoding PIK3R3 can be used to clone genomic DNA encoding PIK3R3 in accordance with established techniques. A portion of the genomic DNA encoding PIK3R3 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PIK3R3 polypeptide.

Nucleic acid encoding the PIK3R3 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The nucleic acid molecules encoding the PIK3R3 polypeptides or fragments thereof described herein are useful for chromosome identification. In this regard, there exists an ongoing need to identify new chromosome markers, since relatively few chromosome-marking reagents, based upon actual sequence data are presently available. Each PIK3R3 nucleic acid molecule of the present invention can be used as a chromosome marker.

The PIK3R3 polypeptides and nucleic acid molecules of the present invention may also be used diagnostically for tissue typing, wherein the PIK3R3 polypeptides of the present invention may be differentially expressed in one tissue as compared to another, preferably in a diseased tissue as compared to a normal tissue of the same tissue type. PIK3R3 nucleic acid molecules will find use for generating probes for PCR, Northern analysis, Southern analysis and Western analysis.

This invention encompasses methods of screening compounds to identify those that mimic the PIK3R3 polypeptide (agonists) or prevent the effect of the PIK3R3 polypeptide (antagonists). Screening assays for antagonist drug candidates are designed to identify compounds that bind or complex with the PIK3R3 polypeptides encoded by the genes identified herein, or otherwise interfere with the interaction of the encoded polypeptides with other cellular proteins, including e.g., inhibiting the expression of PIK3R3 polypeptide from cells. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a PIK3R3 polypeptide encoded by a nucleic acid identified herein under conditions and for a time sufficient to allow these two components to interact.

In binding assays, the interaction is binding and the complex formed can be isolated or detected in the reaction mixture. In a particular embodiment, the PIK3R3 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PIK3R3 polypeptide and drying. Alternatively, an immobilized antibody, e.g., a monoclonal antibody, specific for the PIK3R3 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PIK3R3 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GALA, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for □-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PIK3R3 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the PIK3R3 polypeptide may be added to a cell along with the compound to be screened for a particular activity and the ability of the compound to inhibit the activity of interest in the presence of the PIK3R3 polypeptide indicates that the compound is an antagonist to the PIK3R3 polypeptide. Alternatively, antagonists may be detected by combining the PIK3R3 polypeptide and a potential antagonist with membrane-bound PIK3R3 polypeptide receptors or recombinant receptors under appropriate conditions for a competitive inhibition assay. The PIK3R3 polypeptide can be labeled, such as by radioactivity, such that the number of PIK3R3 polypeptide molecules bound can be used to determine the effectiveness of the potential antagonist. Preferably, expression cloning is employed wherein polyadenylated RNA is prepared from a cell responsive to the PIK3R3 polypeptide and a cDNA library created from this RNA is divided into pools and used to transfect COS cells or other cells that are not responsive to the PIK3R3 polypeptide. Transfected cells that are grown on glass slides are exposed to labeled PIK3R3 polypeptide. The PIK3R3 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for binding identification, labeled PIK3R3 polypeptide can be photoaffinity-linked with cell membrane or extract preparations that express the receptor molecule. Cross-linked material is resolved by PAGE and exposed to X-ray film. The labeled complex containing the bound proteins can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative binding partner.

In another assay for antagonists, mammalian cells or a membrane preparation expressing the receptor would be incubated with labeled PIK3R3 polypeptide in the presence of the candidate compound. The ability of the compound to enhance or block this interaction could then be measured.

More specific examples of potential antagonists include an oligonucleotide that binds to the fusions of immunoglobulin with PIK3R3 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the PIK3R3 polypeptide that recognizes the receptor but imparts no effect, thereby competitively inhibiting the action of the PIK3R3 polypeptide.

Another potential PIK3R3 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PIK3R3 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the PIK3R3 polypeptide. The antisense RNA oligonucleotide hybridizes to the mRNA in vivo and blocks translation of the mRNA molecule into the PIK3R3 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the PIK3R3 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, or other relevant binding site of the PIK3R3 polypeptide, thereby blocking the normal biological activity of the PIK3R3 polypeptide. Examples of small molecules include, but are not limited to, small peptides or peptide-like molecules, preferably soluble peptides, and synthetic non-peptidyl organic or inorganic compounds.

PIK3R3 overexpression or amplification may be evaluated using an in vivo diagnostic assay, e.g., by administering a molecule (such as an RNAi, oligopeptide or small molecule) which binds the molecule to be detected and is tagged with a detectable label (e.g., a radioactive isotope or a fluorescent label) and externally scanning the patient for localization of the label.

As described above, the RNAi and small molecules of the invention have various non-therapeutic applications. The RNAi and small molecules of the present invention can be useful for diagnosis and staging of PIK3R3 polypeptide-expressing cancers (e.g., in radioimaging). The oligopeptides and small molecules are also useful for purification or immunoprecipitation of PIK3R3 polypeptide from cells, for detection and quantitation of PIK3R3 polypeptide in vitro, e.g., in an ELISA or a Western blot, to kill and eliminate PIK3R3-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. RNAi or small molecule therapy may be especially desirable in elderly patients who do not tolerate the toxicity and side effects of chemotherapy well and in metastatic disease where radiation therapy has limited usefulness. The tumor targeting RNAi and small molecules of the invention are useful to alleviate PIK3R3-expressing cancers upon initial diagnosis of the disease or during relapse. For therapeutic applications, the RNAi or small molecule can be used alone, or in combination therapy with, e.g., hormones, antiangiogens, or radiolabelled compounds, or with surgery, cryotherapy, and/or radiotherapy. RNAi or small molecule treatment can be administered in conjunction with other forms of conventional therapy, either consecutively with, pre- or post-conventional therapy. Chemotherapeutic drugs such as TAXOTERE® (docetaxel), TAXOL® (palictaxel), estramustine and mitoxantrone are used in treating cancer, in particular, in good risk patients. In the present method of the invention for treating or alleviating cancer, the cancer patient can be administered RNAi or small molecule in conjunction with treatment with the one or more of the preceding chemotherapeutic agents. In particular, combination therapy with palictaxel and modified derivatives (see, e.g., EP0600517) is contemplated. The RNAi or small molecule will be administered with a therapeutically effective dose of the chemotherapeutic agent. In another embodiment, the RNAi or small molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

Isolated PIK3R3 polypeptide-encoding nucleic acid can be used herein for recombinantly producing PIK3R3 polypeptide using techniques well known in the art and as described herein. In turn, the produced PIK3R3 polypeptides can be employed for generating anti-PIK3R3 antibodies using techniques well known in the art and as described herein.

Antibodies specifically binding a PIK3R3 polypeptide identified herein, as well as other molecules identified by the screening assays disclosed hereinbefore, can be administered for the treatment of various disorders, including cancer, in the form of pharmaceutical compositions.

Internalizing antibodies are preferred as the PIK3R3 polypeptide is intracellular. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### IGF2 is Expressed in a Distinct Group of Glioblastoma Multiformans (GBM)

In the light of the previous studies, the instant inventors used nucleic acid microarrays to perform gene profiling of GBMs to look for other genes that contribute to GBM formation and/or proliferation. Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (for example, disease tissue) sample is greater than hybridization signal of a probe from a control (for example, normal tissue) sample, the gene or genes overexpressed in the disease tissue are identified. The implication of this result is that an overexpressed protein in a disease tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

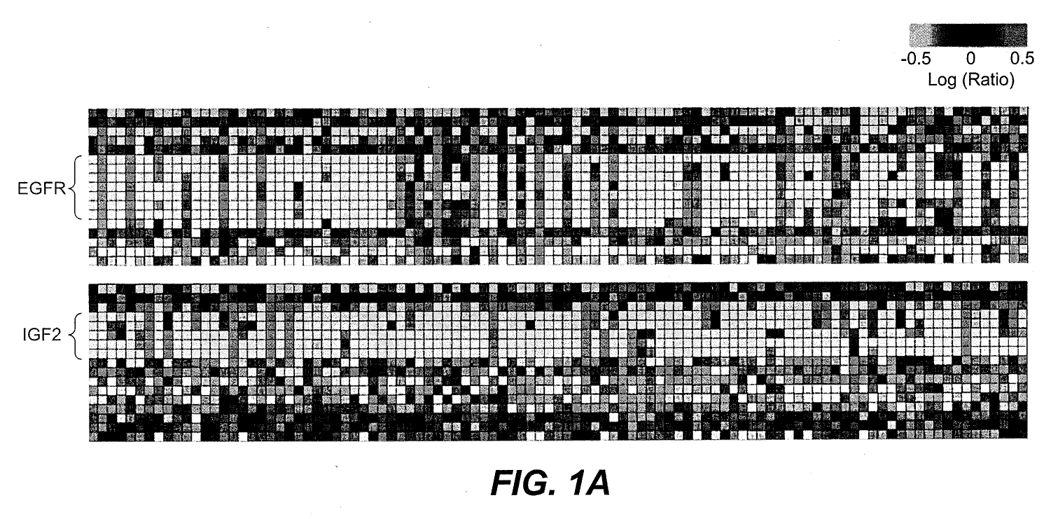

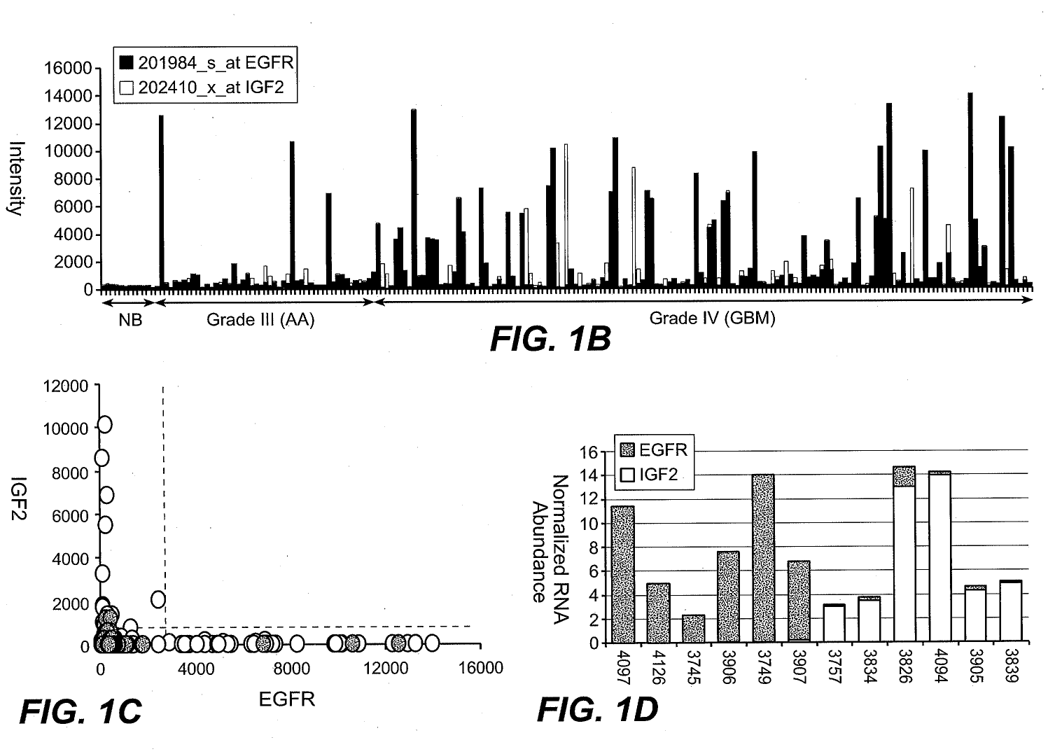

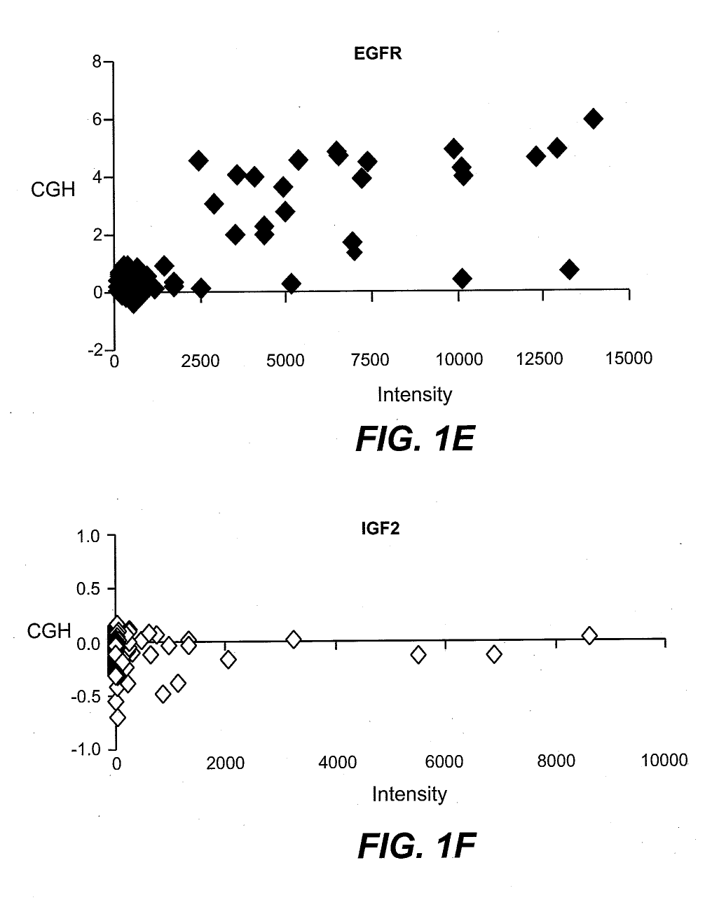

The specific microarray, comparative genomic hybridizations and Taqman assays were performed as previously described and information on histological features and demographics of the cases investigated are as described in Phillips et al., Cancer Cell 9:157-173 (2006). In addition, eight new cases from juvenile patients are included in this study. Table 1, infra, displays sample identifiers for specimens included in the current study along with microarray expression intensity values for EGFR and IGF2. ROSETTA RESOLVER® software (Rosetta Biosoftware, Seattle, Wash. 98109) was used to generate the heat map in FIG. 1. Quantitative analysis of microarray data was performed using signal intensity values from Microarray Analysis Suite version 5 were utilized with a scaling factor of 500. EGFR overexpression was defined as >5 fold increase over the median value of all tumors. For IGF2, a more conservative standard of overexpression was defined as >50 fold increase over the median value of all tumors.

Using this approach, a subset of GBMs was identified which overexpressed IGF2 and was distinct from EGFR expressing tumors. A number of high grade tumors (194 tumors representing 165 cases) were profiled with Affimetrix U133 A and B chips, using 13 normal brain samples as control. Screening of these samples revealed a group of IGF2 expressing GBM distinct from EGFR expressing GBM. In a sample of 44 primary Grade III tumors, EGFR overexpression was seen in 3 cases (7%), while one case (2%) overexpressed IGF2. This is shown graphically in FIGS. 1B and C. No overlap was seen between these expression profiles. Analysis of 121 Grade IV tumors emphasized this exclusivity. Of the 121 GBM cases analyzed, 13% overexpress IGF2, while 28% overexpress EGFR (FIGS. 1B and C). These tumor differences are mutually exclusive, resulting in a statistically significant result of p<0.05, using Fisher's Exact test.

IGF2 overexpression is more robust than EGFR overexpression. Analysis of the microarray results for relative expression levels, show IGF2 overexpression had a maximal increase of 500 fold over normal brain or in IGF2 negative tumors. In contrast, EGFR overexpression had a maximal value of 50 fold above normal brain. These results are shown graphically in FIG. 1B.

The exclusivity of IGF2 GBM to EGFR GBM was confirmed using Taqman analysis, using probes 18 nucleotides in length residing in exons 8 of EGFR and exon 4 of the IGF2 sequence. Taqman was performed on 6 IGF2 overexpressing tumors and on 6 EGFR expressing tumors. Consistent with the microarray data, IGF2 levels were faint to undetectable in EGFR samples, and IGF2 expression was high in non-EGFR expressing samples (FIG. 1D). This data confirms that EGFR and IGF2 are mutually exclusive subsets with consistent relative RNA abundance for each gene. This suggests that IGF2 signaling may support tumor growth in cases that lack the increase in tyrosine kinase activity supplied by EGFR and inhibitors of IGF2 or the IGF2 signaling cascade would be useful in alleviating IGF2 dependent GBM growth.

### Example 2

### Recurrent Tumors Maintain IGF2 Exclusivity

To determine if a primary IGF2 overexpressing tumor maintains this expression profile in a re-occurring tumor, 27 pairs of matched primary and subsequent recurring tumors were analyzed. Of six (6) primary tumors that overexpressed EGFR, five (5) recurring tumors maintained strong EGFR expression. The EGFR negative recurring tumor showed subsequent high expression of IGF2. With regards to IGF2 expression, two (2) IGF2 overexpressing primary tumors also showed IGF2 overexpression upon recurrence of the tumor. This data shows that IGF2 and EGFR are the two main pathways driving formation of some high-grade gliomas. Either pathway is capable of establishing and maintaining tumor growth, but independent of each other. Therefore, antagonists to either pathway would be useful in the formation or recurrence of GBM.

### Example 3

### Genomic Analysis of GBM

Comparative genomic hybridization (CGH), a method for determining the copy number of genes in cells, was performed as described in Phillips et al., Cancer Cell 9:157-173 (2006), based on the protocols published in Misra et al., Genes Chromosomes Cancer 45: 20-30 (2006); Misra et al., Clin. Cancer Res. 11: 2907-18 (2005). Genomic amplification of the copy numbers of genes in a cell can lead to overexpression or unregulated expression of the gene. CGH analysis was performed on a subset of 88 GBM tumors used in microarray profiling. There was correlation between tumors with EGFR amplification and overexpression (FIG. 1E and Table 6). Of the 21 cases showing EGFR amplification, 20 showed appropriate overexpression. Conversely, of 25 cases with EGFR overexpression, 21 cases showed EGFR amplification. With regard to IGF2, no amplification was seen near the IGF2 locus (FIG. 1F). However, the examination of PTEN showed that there was frequent PTEN loss in IGF2 (73%) and EGFR (80%) expressing GBMs. In contrast, tumors negative for IGF2 or EGFR had only modest loss (35%) of PTEN. Differences in PTEN loss among the tumor groups was statistically significant (p<0.05). This data shows that both EGFR and IGF2 have their greatest effect in driving tumor growth when PTEN is absent. Neither IGF2 nor EGFR correlated to chromosomal addition or loss of cell cycle components retinoblastoma (RB), CDK4 or MDM2. However there was good correlation (64%) of EGFR and IGF2 (27%) with loss of p16 (Table 6). Taken in total, the CGH data demonstrates that both IGF2 and EGFR cooperate with PTEN loss to drive cell proliferation by activation of the PI3K/Akt pathway.

TABLE 6
CGH Summary

| | Amplif | Amplif | Loss | Gains | Loss | Loss | Amplif | Amplif |
|---|---|---|---|---|---|---|---|---|
| Expression | PDGFRα | EGFR | PTEN | PIK3R3 | p16 | RB | CDK4 | MDM2 |
| IGF2-OE(n = 11) | 0% | 0% | 64% | 9% | 27% | 27% | 9% | 9% |
| EGFR-OE(n = 25) | 0% | 84% | 80% | 0% | 64% | 20% | 12% | 4% |
| Neither (n = 52) | 10% | 2% | 35% | 10% | 42% | 12% | 12% | 2% |

Table 6. Genomic copy number alterations for 8 genes are presented as percentage of total cases that either overexpress EGFR (EGFR-OE), IGF2 (IGF2-OE) or neither.

### Example 4

### Histological Analysis Confirms IGF2/EGFR Exclusivity

To further validate the molecular differences in the GBM tumors, a sample set of 88 tissue microarrays consisting of cores from GBM tumors were examined using in situ hybridization (ISH) or Immunohistochemistry (IHC). IGF2 ISH was performed according to previously described methods (Philips et al., Endo. 127:965-967 (1990)). The probe consisted of a 873 bp fragment of IGF2 (bp 468-1341 Genbank Accession number NM_{—}000612). Tissues for ISH came from commercial sources including, Cureline (South San Francisco, Calif.), Zymed (South San Francisco, Calif.), Cybridi (Frederick, Md.) and PetaGen (Seoul, South Korea). IGF2 was positive in 6% (5/88) of the GBMs sampled.

IHC was performed on paraffin-embedded section of tumors as previously described (Simmons et al., Can. Res. 61:122-1128 (2001)). Primary antibodies were anti-p-Akt (ser473) from Cell Signaling Technology (Beverly, Mass.), anti-Ki67 (MIB-1, Dako, Carpinteria, Calif.) and anti-EGFR (Dako). Ratings were performed by pathologists blinded to the identity of the specimens. IHC showed EGFR was positive in 48% (41/88) of GBM. Consistent with the microarray data the patterns of expression for each molecule was mutually exclusive.

To examine the histopathology associated with each tumor subtype, 74 full tissue blocks were analyzed. IGF2 was overexpressed in 19% (14/74) of GBM tissue sections examined by ISH when compared to normal surrounding brain tissue. Of the samples positive for IGF2, 7% were rated as highly intense. When the intense samples were tested for EGFR expression via IHC, none of these samples were positive for EGFR staining. Conversely, 20 cases negative for IGF2 were examined for EGFR expression, and 46% showed intense staining for EGFR. This data confirms shows that the two tumor subsets are mutually exclusive.

### Example 5

### IGF2 Overexpression is Associated with Proliferation

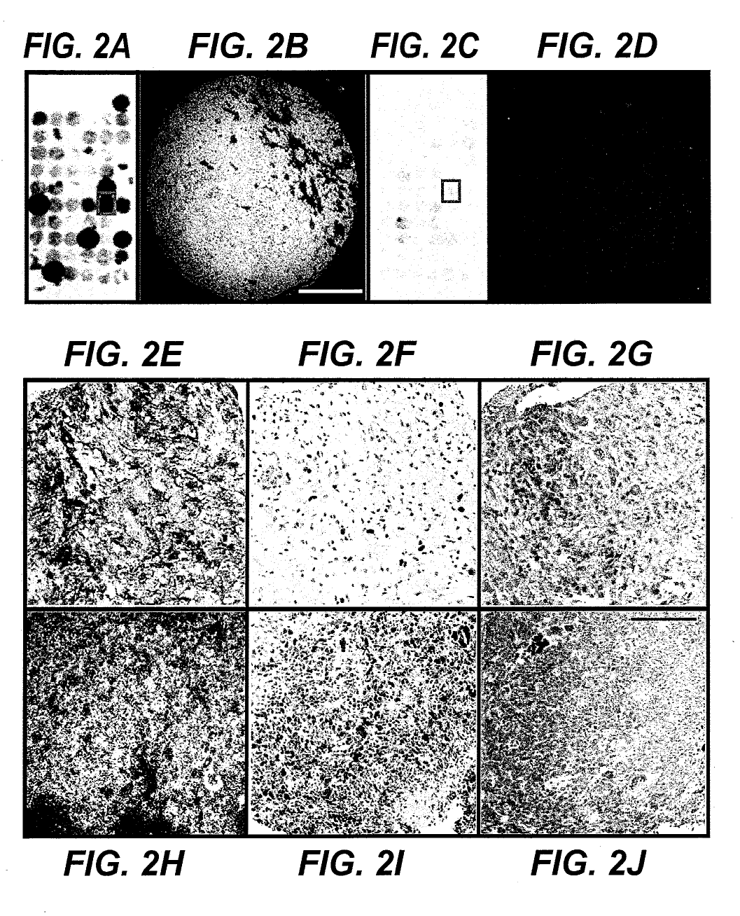

Ki-67 is a commonly used marker of cell proliferation. As shown in FIG. 2B and Table 7, Ki-67 is highly positive in the majority of IGF2 GBMs tested, but is positive only in a minority of the EGFR cases. The mean rating for Ki-67 expression was significantly higher in IGF2 cases than for either EGFR expressing GBM or cases expressing neither receptor. (p<0.05 for both comparisons). In this data set, the activation of PI3K/Akt pathway was also tested. Phospho-Akt was found to be intensely positive in 31% of the EGFR overexpressing GBM, and in 62.5% of the IGF2 overexpressing GBM, as is shown in FIG. 2B and Table 7. The mean intensity of phosphor-Akt staining did not differ for IGF2 or EGFR positive samples. Therefore, IGF2 is associated with GBM that have a high proliferative index as determined by Ki-67 expression. Because IGF2 and PI3K are part of a common signaling pathway it can be inferred from this data that the IGF2/PI3K are a factor driving increased cell proliferation. This increased cell proliferation may be reduced by inhibitors of IGF21PI3K.

TABLE 7
IHC Summary

| | |
|---|---|
| Ki-67++ | p-Akt++ |

Percentage of Positive Cases

| | | |
|---|---|---|
| EGFR++(n = 13) | 23% | 31% |
| IGF2++(n = 8) | 75% | 63% |
| Neither (n = 7) | 29% | 29% |

Mean Ratings for Ki-67 or p-Akt Signal

| | | |
|---|---|---|
| EGFR++ | 0.69 | 1.15 |
| IGF2++ | 1.62* | 1.5 |
| Neither | 0.71 | 1.28 |

Table 7. Summary of IHC and ISH analyses of 28 full-face GBM sections. Ratings for IHC and ISH are made on a 0-2 scale (2-intensely positive, 1-moderately positive, 0- negative). For calculation of percentage of positive cases, only a score of 2 was considered positive.

### Example 6

### IGF2 can Promote the Growth of Glioma Derived Cells

Neurosphere is a term of art used to describe the spheroidal cell clusters that form in vitro when CNS derived cells are cultured in serum free medium. Neurosphere assays were developed initially by Reynolds and Weiss using CNS cells from the mouse striatium, but recently have been used to study neural stem cells (Reynolds et al., J. Neurosci. 12: 4565-4574 (1992) and Ignatova et al., Glia 39:193-206 (2002)). Currently, neurospheres are used in experiments probing the cancer stem cell field, including brain tumor stem cells in human GBM (Ignatova supra and Phillips et al., Cancer Cell 9:157-173 (2006)).

Previously described GBM cell lines were used for the instant in vitro studies (Hartman et al., Internat. J. One. 15: 975-982 (1999)). To create neurosphere cultures, two cell lines (G63 and G69) were sorted using a CD133 cell isolation kit (Miltenyi Biotech) according to manufacturer's instructions, and maintained in culture as described for primary GBM specimens (Singh et al., Nature 432: 396-401 (2004)). All neurosphere cultures were maintained in Neurobasal medium (Invitrogen) with N2 supplement (Invitrogen) and NSF1 (Cambrex). For growth studies, both EGF and IGF2 were added at a concentration of 20 ng/ml. Neurospheres of primary GBM were obtained as unsorted dissociate of primary tumor (from Drs. M. Westphal and K. Lamszuz, Univ. Hamburg), expanded and maintained under the same conditions described for cell line-derived neurospheres. Neurosphere assays were performed in triplicate.

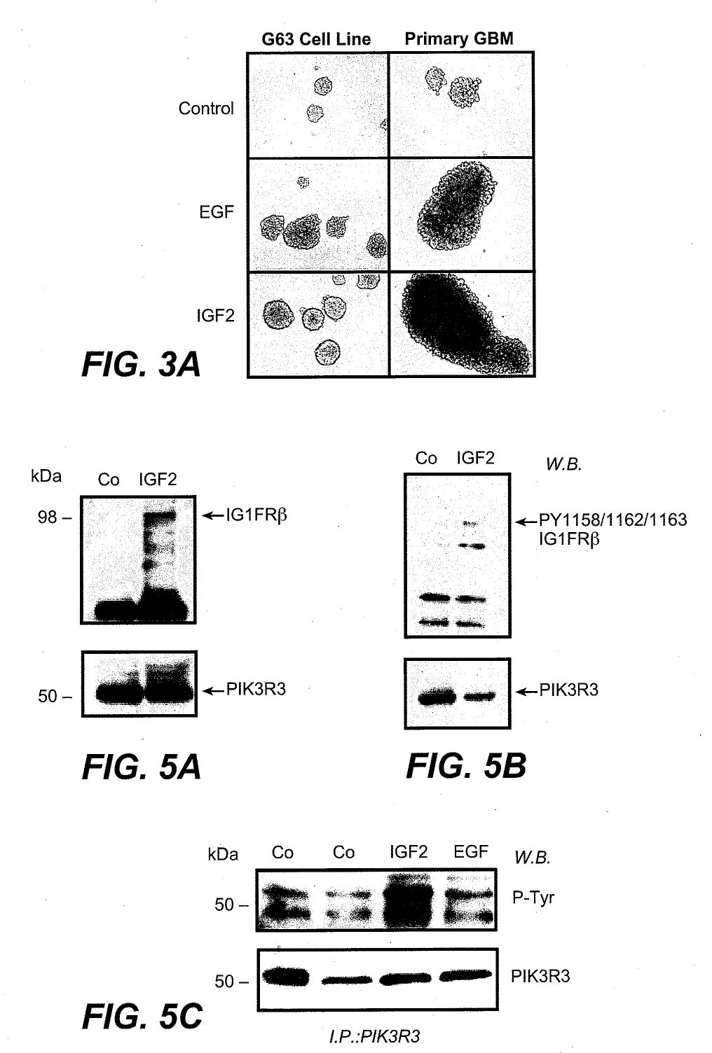

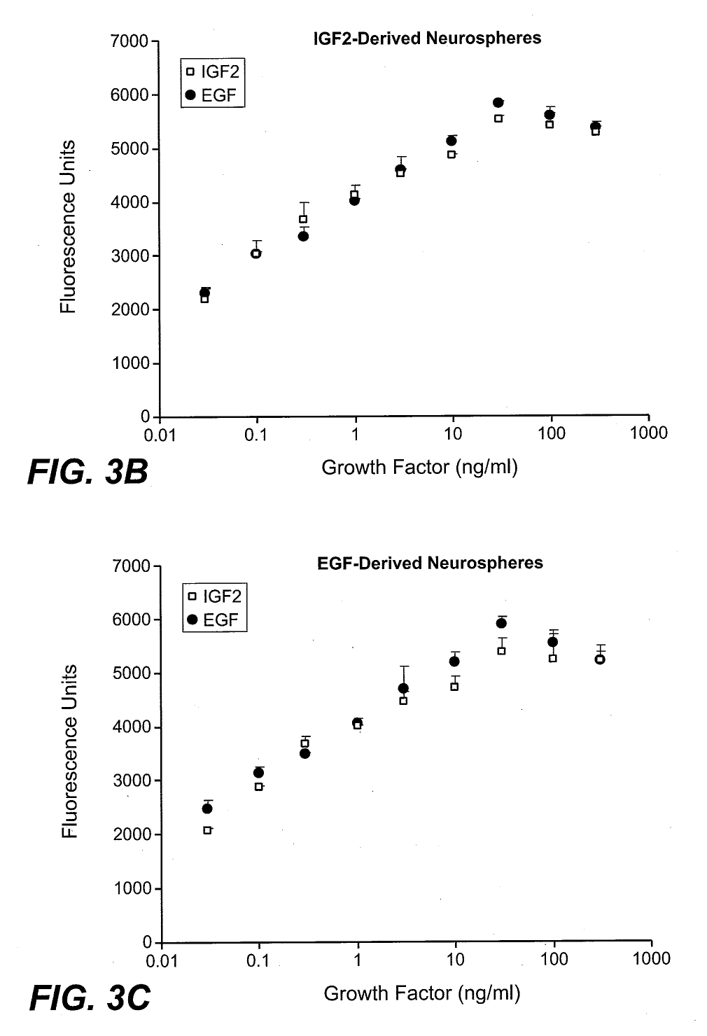

Using the neurosphere system, IGF2 was found to induce a proliferative response that was indistinguishable from the response to EGF (FIG. 3A). Neurospheres failed to grow in the absence of IGF2 or EGF, but proliferated rapidly at a maximal concentration of 20 ng/ml of either factor. When IGF2-dependent neurospheres were disassociated and subjected to a proliferation assay using increasing concentrations of EGF or IGF2, both growth factors induced rapid growth. The growth rate based on growth factor dosage produced an identical growth curve for each growth factor (FIG. 3B). Conversely, when EGF-dependent neurospheres were disassociated, they showed growth rates that were identical after treatment with EGF or IGF2 (FIG. 3C). Cells taken from primary GBM, disassociated and used in the neurosphere culture system also showed rapid growth upon addition of EGF or IGF2 (FIG. 3A). These experiments show that IGF2 and EGF are acting on similar if not identical cell populations within the cultures, and that both factors are effective in promoting GBM cell proliferation. To confirm this result in primary tumors, primary GBM tissue was disassociated and treated with similar concentrations of IGF2 or EGF, and these cells also responded with growth.

To show that IGF2 induced growth was specific, an IGF1R blocking antibody was used to block receptor-ligand interaction. Cell proliferation induced by IGF2 was blocked with 10 ug/ml anti-IGF1R antibody. Neurospheres stimulated with EGF were not affected by the antibody.

These experiments show that IGF2 has a proliferative effect similar to that of EGF and this pathway is as important for cell proliferation, therefore any therapeutic that is able to inhibit the IGF2 pathway will prove useful in the reduction of tumor growth.

### Example 7

### IGF2 and PIK3R3 are Overexpressed in Proliferative GBMs

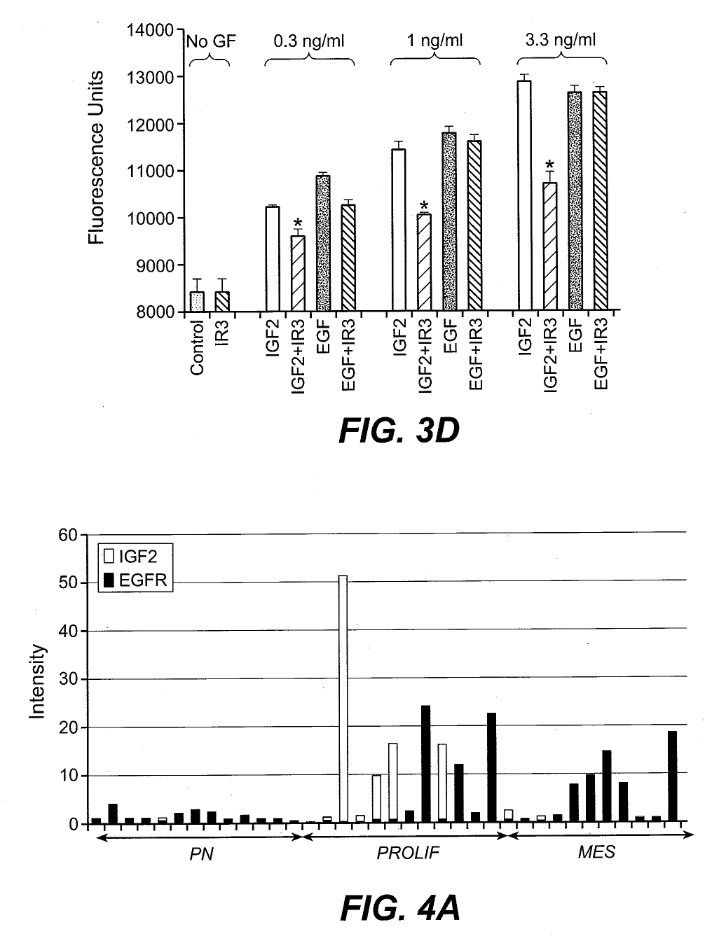

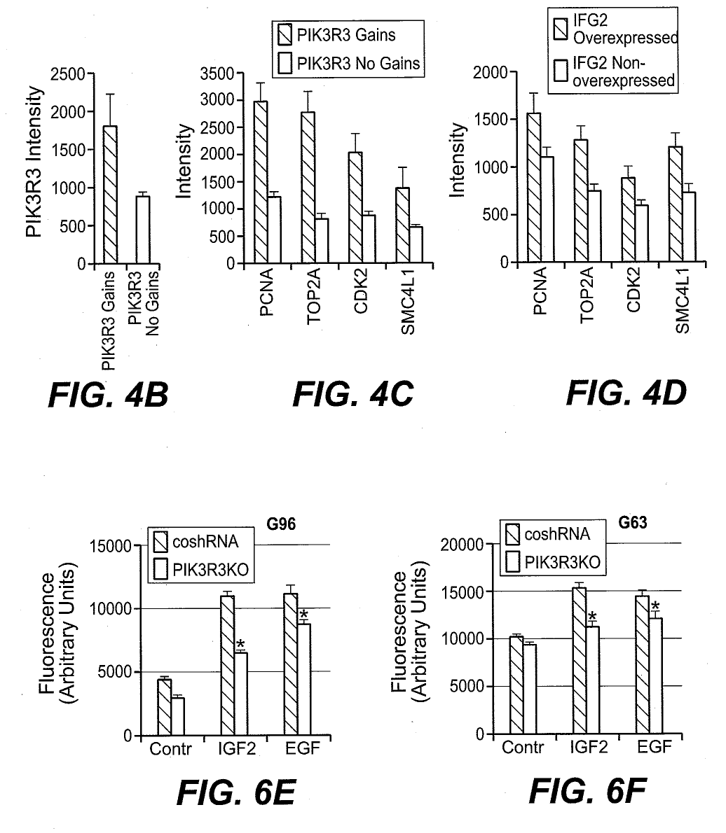

High grade gliomas can be divided up into 3 prognostic subclasses: proneural, proliferative and mesenchymal, each named according to distinct gene signatures (Phillips et al., Cancer Cell 9:157-173 (2006)). A set of 12 samples from each subclass was examined via microarray. IGF2 overexpression was limited to the proliferative subclass, while EGFR overexpression was found in the proliferative and mesenchymal subclasses. When combined with the Ki-67 result that IGF2 induces proliferation, leads to the conclusion that IGF2 signaling is involved in the development of highly proliferative GBM. This hypothesis lead to the investigation of downstream effector molecules of the IGF2 pathway, as activating mutations or amplification of IGF2 effector genes may result in the same aggressive GBM phenotype. Analysis of GBMs showed that PIK3R3 (SwissProt Accession P55G_HUMAN), a subunit of PI3K, had copy number gains at its respective genomic locus. This results in significant PIK3R3 overexpression. GBMs with overexpression of PIK3R3 showed resultingly higher expression of four markers of proliferation (PCNA, TOP2A, CDK2 and SMC4L1) as shown in FIG. 4C. These four markers showed increased expression levels in GBMs overexpressing IGF2, when compared to GBMs negative for IGF2 (FIG. 4D).

PIK3R3 amplification was shown in six GBM cases, with five of these cases lacking either IGF2 or EGFR overexpression. One PIK3R3 amplified GBM was also positive for IGF2 overexpression. Therefore, both IGF2 and PIK3R3 overexpression is associated with a highly proliferative GBM phenotype and antagonists to PIK3R3 function or expression would be useful in alleviating highly proliferative GBMs.

### Example 8

### PIK3R3 is the Mediator of IGF2 Signaling in Human GBM

Because previous results show that IGF2 and PIK3R3 are associated with a highly proliferative GBM phenotype, and given the result that PIK3R3 was cloned using a yeast-two hybrid system with IGFR1 as “bait,” is was necessary to confirm that PIK3R3 mediated IGF2 signal transduction in GBM. This was confirmed by growing the G63 cell line as neurospheres, disassociating the neurospheres, and then allowing the cells to grow an additional 48 hours in serum free/growth factor free media. Recombinant IGF2 (R&D Systems, 292-G2)) was then added at a concentration of 20 ng/ml and allowed to stimulate the cells for 15 minutes. After this time period, the cells were washed and lysed. The cell lysates were immunoprecipitated with Protein-G (Pierce Biotech, Rockford, Ill.) coupled to anti-PIK3R3 antibody (Ab-253-2, Abcam, plc, Cambridge, UK), washed, boiled for 5 minutes in loading buffer (Pierce) and resolved on SDS-PAGE gens. Western blotting was performed using anti-IGF1Rβ antibody (SC-713, Santa Cruz), or a phospho-specific anti-IGF1Ra antibody (anti-ppIGF1R/Y1162/Y1163, Novus Biologicals). Anti-phosphotyrosine (Upstate, 4G10) antibody was used to detect tyrosine phosphorylated proteins, e.g., PIK3R3. Anti-actin acting antibody was purchased from Abcam (Ab-8277). pAkt (Ser 473) was detected using Cell Signaling 193H12 antibody, and total Akt antibody was obtained from Cell Signaling.

The immunoprecipitations showed that endogenous PIK3R3 physically associates with IGF1R (FIG. 5A). This data also indicated that phosphorylated IGF1R was specifically associated with PIK3R3 in IGF2 stimulated cells. A physical complex was formed that includes tyrosine phosphorylated PIK3R3 upon stimulation with IGF2 (20 ng/ml), EGF (10 ng/ml) or insulin (FIG. 5C). In conclusion, this data supports a pathway model that upon stimulation of IGF2, PIK3R3 is recruited by phospho-IGF1R, becomes activated by phosphorylation and mediates downstream events, such as the phosphorylation of Akt.

### Example 9

### RNAi Knockdown of PIK3R3 Inhibits IGF2 and EGF Dependent Cell Proliferation

In order to confirm the role of PIK3R3 in cell proliferation, short inhibitory RNAs (RNAi) were synthesized to reduce or “knockdown” the amount of PIK3R3 in the cell. ShRNA constructs were purchased from Open Biosystems. Several constructs were tested in transient transfections, and two of them were used in generating stable cell lines: RHS1764-9494180 for G96 and RHS164-9208343 for G63. Phoenix Ampho (ATCC SD3443, retroviral producer line, MMUL-based) cells were used for retroviral production and virus-containing supernatant supplemented with 5 μg/ml polybrene were used to infect glioma cells. Puromycin resistant colonies were selected and polled clones were used for subsequent experiments. Neurospheres from CD133-sorted cells of both G63 and G96 were tested for growth responses.

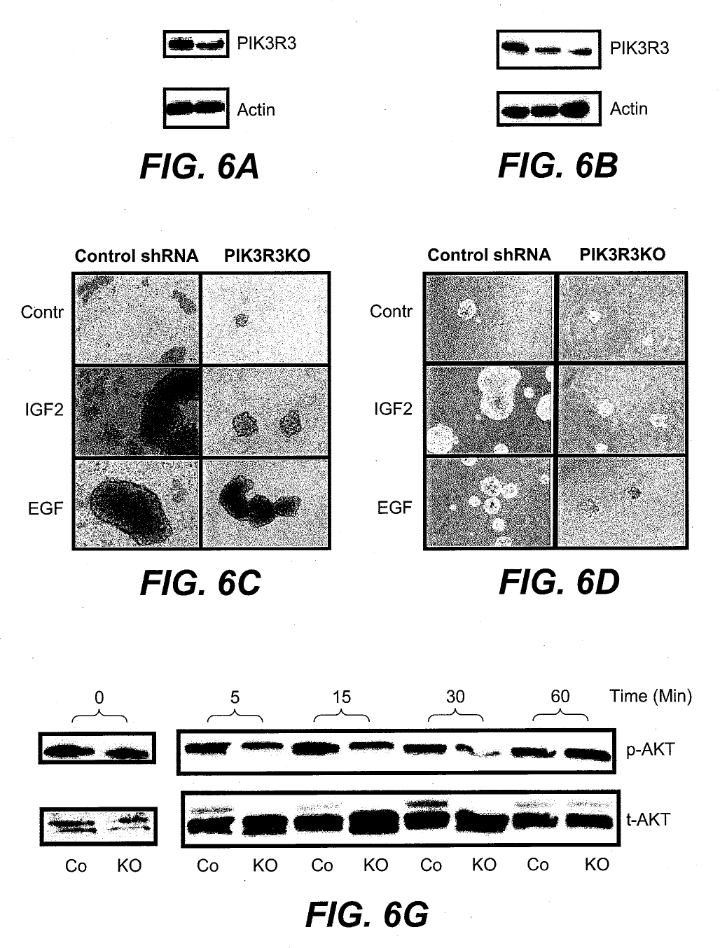

Glioma cells derived from neurospheres (G63, G96) grown in neural basal medium supplemented with N2 and NSF (Cambrex) were stimulated every 24 h for 3 days with increasing concentrations of EGF or IGF2. Where indicated, the α-IR3 IGF1R blocking antibody (Calbiochem, #GR11L) was added to cultures 1 hour prior to growth factor stimulation. Alamar blue (Trek Biodiagnostic) was used to assess cell viability. Cell viability assays described in FIG. 6 were performed using dissociated neurospheres 14 days after initial plating. All experiments were repeated at least three times for each cell line.

Neurospheres were stably transfected (replicated in triplicate) with PIK3R3 RNAi expression constructs, resulting in a knockdown of PIK3R3 mRNA by 81% in G96 cells and by 85% in G63 cells. This reduction was confirmed at the protein level (as described previously) by Western blotting (FIGS. 6A and 6B). PIK3R3 RNAi stable lines were cell sorted for the marker CD133 and then assayed for cell proliferation upon stimulation with IGF2 or EGF. FIGS. 6C and 6D show that knockdown of PIK3R3 results in reduced cell proliferation and subsequently reduced neurosphere growth in the presence of IGF2 or EGF. Cell proliferation in response to EGF was inhibited by 14% in G96 PIK3R3 RNAi lines and by 35% in G63 PIK3R3 RNAi lines. However, the most dramatic result was seen with IGF2 stimulated cells. IGF2 stimulated PIK3R3 RNAi cell lines showed a reduction in proliferation of 53% for the G96 cells and 64% in the G63 cells. In contrast, in the absence of these growth factors, the PIK3R3 RNAi transfected cells showed only minimal effects on cell proliferation.

Because Akt is a downstream target of PIK3R3, the PIK3R3 RNAi containing lines were used to study the effects on Akt. PIK3R3 RNAi containing G96 cells were grown in neurosphere conditions; growth factors were removed for 48 hours, and then stimulated with IGF2. PIK3R3 RNAi lines stimulated with IGF2 for 5, 15 and 30 minutes show decreased Akt phosphorylation (FIG. 6G). This result was confirmed in G63 PIK3R3 RNAi cell lines. Control kinases such as MAPK showed no change (data not shown).

This data supports the conclusion that PIK3R3 is a key mediator of cell proliferative effects exerted by IGF2 on glioma derived neurospheres. Therefore, inhibitors of PIK3R3 would be useful in alleviating tumor burden of growing gliomas by inhibiting the IGF2-PIK3R3-Akt pathway. The results here have shown that the IGF2 pathway is unique to a certain subset of GBM, and the data would support that inhibitors to the IGF2 pathway components would inhibit tumor growth and progression in this GBM subset. Recent trends in cancer biology propose that inhibition of multiple members of the responsive pathway may be the most efficacious. In the instant case, inhibitors to IGF2, PIK3R and Akt may prove more effective than a single inhibitor.

### Example 10

### Microarray Analysis to Detect Upregulation of PIK3R3Polypeptides in Cancerous Glioma Tumors

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (disease tissue) sample is greater than hybridization signal of a probe from a control (normal tissue) sample, the gene or genes overexpressed in the disease tissue are identified. The implication of this result is that an overexpressed protein in a diseased tissue is useful not only as a diagnostic marker for the presence of the disease condition, but also as a therapeutic target for treatment of the disease condition.

The methodology of hybridization of nucleic acids and microarray technology is well known in the art. In the present example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

### Example 11

### Quantitative Analysis of PI3KR3 mRNA Expression

In this assay, a 5′ nuclease assay (for example, TaqMan®) and real-time quantitative PCR (for example, ABI Prizm 7700 Sequence Detection System® (Perkin Elmer, Applied Biosystems Division, Foster City, Calif.)), is used to find genes that are significantly overexpressed in a cancerous glioma tumor or tumors as compared to other cancerous tumors or normal non-cancerous tissue. The 5′ nuclease assay reaction is a fluorescent PCR-based technique which makes use of the 5′ exonuclease activity of Taq DNA polymerase enzyme to monitor gene expression in real time. Two oligonucleotide primers (whose sequences are based upon the gene or EST sequence of interest) are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the PCR amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative and quantitative interpretation of the data. This assay is well known and routinely used in the art to quantitatively identify gene expression differences between two different human tissue samples, see, e.g., Higuchi et al., Biotechnology 10:413-417 (1992); Livak et al., PCR Methods Appl., 4:357-362 (1995); Heid et al., Genome Res. 6:986-994 (1996); Pennica et al., Proc. Natl. Acad. Sci. USA 95(25):14717-14722 (1998); Pitti et al., Nature 396(6712):699-703 (1998) and Bieche et al., Int. J. Cancer 78:661-666 (1998).

The 5′ nuclease procedure is run on a real-time quantitative PCR device such as the ABI Prism 7700™ Sequence Detection. The system consists of a thermocycler, laser, charge-coupled device (CCD) camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

The starting material for the screen is mRNA isolated from a variety of different cancerous tissues. The mRNA is quantitated precisely, e.g., fluorometrically. As a negative control, RNA is isolated from various normal tissues of the same tissue type as the cancerous tissues being tested. Frequently, tumor sample(s) are directly compared to “matched” normal sample(s) of the same tissue type, meaning that the tumor and normal sample(s) are obtained from the same individual.

5′ nuclease assay data are initially expressed as Ct, or the threshold cycle. This is defined as the cycle at which the reporter signal accumulates above the background level of fluorescence. The □Ct values are used as quantitative measurement of the relative number of starting copies of a particular target sequence in a nucleic acid sample when comparing cancer mRNA results to normal human mRNA results. As one Ct unit corresponds to 1 PCR cycle or approximately a 2-fold relative increase relative to normal, two units corresponds to a 4-fold relative increase, 3 units corresponds to an 8-fold relative increase and so on, one can quantitatively and quantitatively measure the relative fold increase in mRNA expression between two or more different tissues. In this regard, it is well accepted in the art that this assay is sufficiently technically sensitive to reproducibly detect an at least 2-fold increase in mRNA expression in a human tumor sample relative to a normal control.

### Example 12

### In Situ Hybridization

In situ hybridization is a powerful and versatile technique for the detection and localization of nucleic acid sequences within cell or tissue preparations. It may be useful, for example, to identify sites of gene expression, analyze the tissue distribution of transcription, identify and localize viral infection, follow changes in specific mRNA synthesis and aid in chromosome mapping.

In situ hybridization is performed following an optimized version of the protocol by Lu and Gillett, Cell Vision 1:169-176 (1994), using PCR-generated ³³P-labeled riboprobes. Briefly, formalin-fixed, paraffin-embedded human tissues are sectioned, deparaffinized, deproteinated in proteinase K (20 g/ml) for 15 minutes at 37° C., and further processed for in situ hybridization as described by Lu and Gillett, supra. A [³³-P] UTP-labeled antisense riboprobe are generated from a PCR product and hybridized at 55° C. overnight. The slides are dipped in Kodak NTB2 nuclear track emulsion and exposed for 4 weeks.

### ³³P-Riboprobe Synthesis

6.0 □l (125 mCi) of ³³P-UTP (Amersham BF 1002, SA<2000 Ci/mmol) were speed vac dried. To each tube containing dried ³³P-UTP, the following ingredients were added:
2.0 □l 5× transcription buffer

### 1.0 □l DTT (100 mM)

2.0 □l NTP mix (2.5 mM: 10□; each of 10 mM GTP, CTP & ATP+10 □l H₂O)

### 1.0 □l UTP (50 □M)

### 1.0 □l Rnasin

1.0 □l DNA template (1 □g)

### 1.0 □l H₂O

1.0 □l RNA polymerase (for PCR products T3=AS, T7=S, usually)

The tubes are incubated at 37° C. for one hour. 1.0 □l RQ1 DNase is added, followed by incubation at 37° C. for 15 minutes. 90 □l TE (10 mM Tris pH 7.6/1 mM EDTA pH 8.0) are added, and the mixture was pipetted onto DE81 paper. The remaining solution is loaded in a Microcon-50 ultrafiltration unit, and spun using program 10 (6 minutes). The filtration unit is inverted over a second tube and spun using program 2 (3 minutes). After the final recovery spin, 100 □l TE is added. 1 □l of the final product is pipetted on DE81 paper and counted in 6 ml of Biofluor II.

The probe is run on a TBE/urea gel. 1-3 □l of the probe or 5 □l of RNA Mrk III is added to 3 □l of loading buffer. After heating on a 95° C. heat block for three minutes, the probe is immediately placed on ice. The wells of gel are flushed, the sample loaded, and run at 180-250 volts for 45 minutes. The gel is wrapped in saran wrap and exposed to XAR film with an intensifying screen in −70° C. freezer one hour to overnight.

### ³³P-Hybridization

### A. Pretreatment of Frozen Sections

The slides are removed from the freezer, placed on aluminium trays and thawed at room temperature for 5 minutes. The trays are placed in 55° C. incubator for five minutes to reduce condensation. The slides are fixed for 10 minutes in 4% paraformaldehyde on ice in the fume hood, and washed in 0.5×SSC for 5 minutes, at room temperature (25 ml 20×SSC+975 ml SQ H₂O). After deproteination in 0.5 □g/ml proteinase K for 10 minutes at 37° C. (12.5 □l of 10 mg/ml stock in 250 ml prewarmed RNase-free RNAse buffer), the sections are washed in 0.5×SSC for 10 minutes at room temperature. The sections are dehydrated in 70%, 95%, 100% ethanol, 2 minutes each.

### B. Pretreatment of Paraffin-Embedded Sections

The slides are deparaffinized, placed in SQ H₂O, and rinsed twice in 2×SSC at room temperature, for 5 minutes each time. The sections are deproteinated in 20 □g/ml proteinase K (500 □l of 10 mg/ml in 250 ml RNase-free RNase buffer; 37° C., 15 minutes)—human embryo, or 8× proteinase K (100 □l in 250 ml Rnase buffer, 37° C., 30 minutes)—formalin tissues. Subsequent rinsing in 0.5×SSC and dehydration are performed as described above.

### C. Prehybridization

The slides are laid out in a plastic box lined with Box buffer (4×SSC, 50% formamide)—saturated filter paper.

### D. Hybridization

1.0×10⁶ cpm probe and 1.0 □l tRNA (50 mg/ml stock) per slide are heated at 95° C. for 3 minutes. The slides are cooled on ice, and 48 □l hybridization buffer are added per slide. After vortexing, 50 □l ³³P mix are added to 50 □l prehybridization on slide. The slides are incubated overnight at 55° C.

### E. Washes

Washing is done 2×10 minutes with 2×SSC, EDTA at room temperature (400 ml 20×SSC+16 ml 0.25M EDTA, V_{f}=4 L), followed by RNaseA treatment at 37° C. for 30 minutes (500 □l of 10 mg/ml in 250 ml Rnase buffer=20 □g/ml). The slides are washed 2×10 minutes with 2×SSC, EDTA at room temperature. The stringency wash conditions can be as follows: 2 hours at 55° C., 0.1×SSC, EDTA (20 ml 20×SSC+16 ml EDTA, V_{f}=4 L).

### F. Oligonucleotides

In situ analysis is performed on a variety of DNA sequences disclosed herein. The oligonucleotides employed for these analyses is obtained so as to be complementary to the nucleic acids (or the complements thereof) as shown in the accompanying figures.

### Example 13

### Preparation of Antibodies that Bind PIK3R3

Techniques for producing monoclonal antibodies are known in the art and are described, for instance, in Goding, supra. Immunogens that may be employed include purified PIK3R3 polypeptides, fusion proteins containing GDM polypeptides, and cells expressing recombinant PIK3R3 polypeptides on the cell surface. Selection of the immunogen can be made by the skilled artisan without undue experimentation.

Mice, such as Balb/c, are immunized with the above immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-GDM antibodies.

After a suitable antibody titer has been detected, the animals “positive” for antibodies can be injected with a final intravenous injection of GDM polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells are screened in an ELISA for reactivity against PIK3R3. Determination of “positive” hybridoma cells secreting the desired monoclonal antibodies against PIK3R3 is within the skill in the art.

The positive hybridoma cells can be injected intraperitoneally into syngeneic Balb/c mice to produce ascites containing the anti-PIK3R3 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 14

### Tumor Screening

Antagonists to PIK3R3 polypeptides may be determined in vivo by a nude mouse model. Mammalian cells can be transfected with sufficient amounts of PIK3R3 polypeptide expressing plasmid to generate high levels of PIK3R3 polypeptide in the cell line. A known number of overexpressing cells can be injected sub-cutaneously into the flank of nude mice. After allowing sufficient time for a tumor to grow and become visible and measurable (typically 2-3 mm in diameter), the mice can be treated with a potential PIK3R3 antagonist. To determine if a beneficial effect has occurred, the tumor is measured in millimeters with Vernier calipers, and the tumor burden is calculated; Tumor weight=(length×width²)/2 (Geran, et al., Cancer Chemotherapy Rep., 3: 1-104 (1972). The nude mouse tumor model is a reproducible assay for assessing tumor growth rates and reduction of tumor growth rate by a possible anti-tumor agent in a dose dependant manner. As an example, the compound 317615-HCL, a candidate Protein Kinase C□ inhibitor, was found to have an anti-tumor effect using this model Teicher et al., Can. Chemo. Pharm. 49: 69-77 (2002).

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. A method for inhibiting the growth of a glioma tumor that expresses a PIK3R3 polypeptide, wherein the growth of said glioma tumor is at least in part dependent upon the growth potentiating effect(s) of a PIK3R3 polypeptide, wherein the method comprises contacting a cell of the glioma tumor with an effective amount of a PIK3R3 antagonist.

2. The method of claim 1, wherein the glioma tumor does not overexpress an EGFR polypeptide.

3. The method of claim 1, wherein the glioma tumor overexpresses an IGF2 polypeptide.

4. The method of claim 1, wherein Akt/PIK3 signaling in the glioma tumor is antagonized.

5. The method of claim 1, wherein the growth is completely inhibited.

6. The method of claim 1, wherein the growth inhibition results in the death of the cell.

7. The method of claim 1, wherein the PIK3R3 antagonist is a PIK3R3 small molecule antagonist.

8. The method of claim 1, wherein the PIK3R3 antagonist binds to nucleic acid encoding the PIK3R3 polypeptide.

9. The method of claim 8, wherein the nucleic acid is RNA.

10. The method of claim 9, wherein the PIK3R3 antagonist is a PIK3R3 RNAi.

11. The method of claim 1, which further comprises contacting the glioma tumor with an effective amount of an Akt antagonist prior, after or simultaneously with the PIK3R3 antagonist.

12. The method of claim 11, wherein the Akt antagonist is an antagonist of the catalytic or regulatory domain of PIK3 kinase.

13. The method of claims 1 or 11, which further comprises contacting the glioma tumor with an effective amount of an IgF2 antagonist prior, after or simultaneously with the PIK3R3 antagonist.

14. A method of treating a glioma tumor in a mammal, wherein said tumor expresses a PIK3R3 polypeptide, wherein the method comprises administering to the mammal a therapeutically effective amount of a PIK3R3 antagonist.

15. The method of claim 14, wherein the glioma tumor does not overexpress an EGFR polypeptide.

16. The method of claim 14, wherein the glioma tumor overexpresses an IgF2 polypeptide.

17. The method of claim 14, wherein Akt/PIK3 signaling in the glioma tumor is antagonized.

18. The method of claim 14, wherein the administration of PIK3R3 antagonist results in the reduced growth or shrinkage in growth of the tumor.

19. The method of claim 14, wherein the administration of PIK3R3 antagonist results in the death of the tumor.

20. The method of claim 14, wherein the PIK3R3 antagonist is a PIK3R3 small molecule antagonist.

21. The method of claim 14, wherein the PIK3R3 antagonist binds to the nucleic acid encoding the PIK3R3 polypeptide.

22. The method of claim 21, wherein the nucleic acid is RNA.

23. The method of claim 22, wherein the PIK3R3 antagonist is a PIK3R3 RNAi.

24. The method of claim 14, which further comprises administering a therapeutically effective amount of an Akt antagonist prior, after or simultaneously with the PIK3R3 antagonist.

25. The method of claim 24, wherein the Akt antagonist is an antagonist of the catalytic or regulatory domain of PIK3 kinase.

26. The method of claims 14 or 24, which further comprises contacting the glioma tumor with an effective amount of an IgF2 antagonist prior, after or simultaneously with the PIK3R3 antagonist.

27. A method of diagnosing the presence of a glioma tumor in a mammal, wherein the method comprises comparing the level of expression of PIK3R3 polypeptide or nucleic acid encoding a PIK3R3 polypeptide (a) in a test sample of glioma tissue obtained from said mammal suspected of being cancerous, and (b) in a control sample of known normal cells of the same tissue origin, wherein a higher level of expression of the PIK3R3 polypeptide or nucleic acid encoding PIK3R3 polypeptide in the test sample, as compared to the control sample, is indicative of the presence of a glioma tumor in the mammal from which the test sample was obtained.

28. The method of claim 27, wherein the nucleic acid is DNA.

29. The method of claim 27, wherein the nucleic acid is RNA.

30. The method of claim 27, wherein the expression of PIK3R3 polypeptide expression is measured by a reagent selected from the group consisting of: anti-PIK3R3 antibody, anti-PIK3R3-binding antibody fragment, PIK3R3 binding oligopeptide and PIK3R3 small molecule.

31. The method of claim 27, wherein the expression of nucleic acid encoding PIK3R3 polypeptide is measured by a reagent selected from the group consisting of: PIK3R3 antisense oligonucleotide and PIK3R3 RNAi.

32. A method for diagnosing the severity of a glioma tumor in a mammal, wherein the method comprises: (a) contacting a test sample comprising cells from said glioma tumor or extracts of DNA, RNA, protein or other gene product(s) obtained from the mammal with a reagent that binds to the PIK3R3 polypeptide or nucleic acid encoding PIK3R3 polypeptide in the sample, (b) measuring the amount of complex formation between the reagent with the PIK3R3-encoding nucleic acid or PIK3R3 polypeptide in the test sample, wherein the formation of a high level of complex, relative to the level in known healthy sample of similar tissue origin, is indicative of an aggressive tumor.

33. The method of claim 32, wherein the method further comprises determining if the glioma tumor does not overexpress an EGFR polypeptide.

34. The method of claim 32, wherein the method further comprises determining if the glioma tumor overexpresses an IGF2 polypeptide.

35. The method of claim 32, wherein the reagent is selected from the group consisting of: anti-PIK3R3 antibody, PIK3R3-binding antibody fragment, PIK3R3 binding oligopeptide, PIK3R3 small molecule, PIK3R3 nucleic acid, PIK3R3 RNAi and PIK3R3 antisense oligonucleotide.

36. A method of screening for PIK3R3 antagonists, comprising (a) contacting a test sample of PIK3R3 expressing glioma cells with a test compound and (b) comparing the expression of PIK3R3 in the contacted cells with control glioma cells that have not been contacted; wherein a lowered expression level in the contacted cells is indicative of a PIK3R3 antagonist, and a therapeutic for the treatment of glioma tumors.

37. A composition comprising pharmaceutically-acceptable carrier, excipient or stabilizer and therapeutically effective amounts of (i) a PIK3R3 antagonist in combination with (ii) an IGF2 antagonist, with optionally (iii) an Akt antagonist.

38. The composition of claim 37, wherein the PIK3R3 antagonist is selected from the group consisting of: a PIK3R3 binding oligopeptide, PIK3R3 small molecule and PIK3R3 RNAi.

39. An article of manufacture comprising a container and a PIK3R3 antagonist contained within the container, wherein the PIK3R3 antagonist and instructions for use of the PIK3R3 antagonist in the therapy, diagnosis and/or prognosis or glioma.

40. The article of manufacture of claim 39, wherein the instructions are in the form of a label affixed to the container or a package insert included within the container.

41. The article of manufacture of claim 40, further comprising an IGF2 antagonist, and optionally an Akt-antagonist.


SEQUENCE LISTING (2 sequences)

SEQ ID NO: 1 - DNA, 5621 bp, Homo sapiens
gtggctggagtccgggcagagcttgagggcagttggtgcggtcgggttggttcttacaccccggcgggagcgcccagacaagccgagctgactggacttctccggccggccccattcccgaggctgcggcagcttcggttccgagaccgaccggagaggagcccgagtcccggcctctgggggattcgctctctgcagaccagtgggaccccgaaacttgaacgcaatctccagcccccttttttgccttcctttgtcacttgcccgggtttctcccaacgtgttcttttttttcctcttcattctccctccttcgaaggacacaaaagtggcttccgcggaaagatttggaggcggtgggagcttttctccccggagagcgactgtgtagaaaggatttttgggaagccgctttttaacacctctgctctccgtcccccaagcctctgtgtaatcctctgaggagaaaagcccatagcttgaaagttcgggggcattttgttgtgttctgtaggagagagggggaggaccctgttcgggtagtttggccggactggtactggccgttggaaaacccgaagtacatttccgtgtggaacttttgcagatatatatttttagatttttaaataccagataaaaaatatatgccttctatatatctcctggcgacctgcccctgacagcgcgatgtacaatacggtgtggagtatggaccgcgatgacgcagactggagggaggtgatgatgccctattcgacagaactgatattttatattgaaatggatcctccagctcttccaccaaagccacctaagccaatgacttcagcagttccaaatggaatgaaggacagttctgtttctcttcaggatgcagaatggtactggggggatatttcaagggaggaggtaaatgacaaattgcgggatatgccagatgggaccttcttggtccgagatgcctcaacaaaaatgcagggagattatactttgactttgcggaagggaggcaataataagttaataaagatctatcaccgggatggtaaatatggcttttctgatcctctgacatttaattccgtggtggagctcattaaccactatcaccatgaatctcttgctcagtacaatcccaaacttgatgtgaagctgatgtacccagtgtccagataccaacaggatcagttggtaaaagaagataatattgatgcagtaggtaaaaaactgcaagaataccactctcagtatcaggagaagagtaaagagtatgataggctgtatgaagaatatactagaacatcccaggaaatacagatgaagaggactgcaatagaagcttttaatgaaacaattaaaatatttgaagagcagtgtcacacacaagaacaacatagcaaagaatatattgagcgatttcgcagagaggggaatgaaaaggagattgaacgaattatgatgaattatgataaattgaaatcacgtctgggtgagattcatgatagcaaaatgcgtctagagcaggatttgaagaatcaagctttggacaaccgagaaatagataaaaaaatgaatagcatcaaacctgacctgatccagctgcgaaagatccgagatcaacaccttgtatggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcagataaagaggaagtgggaagagaggtggttctctggcatttttttctacagtttttattagactacgatgagggcattctttctacatagactgcttgttttgcacaagaagtgattttgtgaatgtgaagtggagaggccgagcagcagccggccgggatgggggcattagaggcctgaggttctctaggactcagccatgccgctgcactgacatactaagctggaagcagatgttttttttgaaagtctgtttcattggggtttttgttttgtttagccagacaccctcaacagaatattaggcttgatggttatagcgggtggggttgtatttggaagcctctgaagagaccatgtctttttaaaatctaactcttgagagtgcagcaggggcatggctctgctgggagttgtgttttgctttggcagtctctcttccccccacgaagaaggctgtttaggttttgtgatagaatgggatttgatgaaaaagacaaccaaaggaaaatggggaggcttgggatttcatttaaataatctaagccaagatgataaaaaaaaccttcaactgaaggtattttgtttcttaccaacataatttaggcttcagcatctcaccagcccctccctctgaagaagtattatgttcagaagccaacaaaacagtttgttgccagaccaatgtttgatgggaaaacgtggcactcatagttgaatgtatacttctgtaccaaaacttgaacataaaaagactagaatttgtgagttttagcaaacgctaaaattgatcactgtaactaaccccttctgtccttcctgcctgtttctctgagatgaggaatagcattctttttgtggggatggtgagctttgaatcataaaatgaagttggtgcttgtatggtgtttccttagcctaaagaatgatctgttgtttgaaacctttgtaacttgtttgtatgagtaaagaaaaggtgcaatgcagtgcttttagatggcttgatataccaaataacaatatagaacaacattattatatgtgcttccccaagtttaaaggccctgcagaaatagtaaacatggtttaatttcccttcatttccccctcctttgctggatggggttttgggagctataggttgctaaggaggggagtcagattgtggtcaggtgcctcagtaaatcacagacccaggggccctgtggtccagggtgagagtcacaccacattacacatgtgcttccatacagtggtttctgaagcttttgcagggagagaagatggcttagtgtttagactgttagtagaagccatctggaagcttttctctttgcctttttttgtgatcctgccattaaggctatgtgcagtctgccctcctgctccagtggccttgattttaggccaggaatcttctgctccatgtggcttaagccttccagctgagtgaagctaggcaaatggagtgggggcaggcatctattcctgcccccatcatgccccacacccatcagtcaacactcatttgacaaatagagtccagctgcctctgagccaatcctgggaccataataggctcagagtgagtcagctgtttgaacccaaagctgtgcagtcaggcaccctggctgagctagagaagccagtacctgcatcttggtttataaggcttacagctaggaaagctctagttctgggggtgaaagaaaaatattagtttacctgagcacttatttccatgacagggtctaaaatatggaccatgatgtagacacagatttttaattttggaaaaacctccagttactagtgacgaggatagaaagggaagtgcttctctttggctttttcttggttacactgcagtttcaggattgggtgagacagagacaaatgaacccccctctaaagtcatttaactaatagccagcacatcccttccccaaactgtcaattgaaatcttaactgaaagttttactgaataataccaagctaattgctgttgggcacacctggatggctttgcacctggtgttgaacctgctgaagcaggtggatgctcaagattacgtgcaaggaatccctcccatctggtactaaaatttcagtgtgttctgagtgtcttttaaaccaaaatggaaatacagatacagggctgtagtattcagtaatgtgtctgctccttgttgggcagacaccagcggtgtgcagggagagaccaagtaccatctttatctacacttgggctggcttgtggagaagggctgcttttttcagtcctacattccttcattttttttttcattcttgaattcattgttttgtgggatctaagacccaggggtcatttgagaggtttgacagtatcttttctgaccagttgccacatgacttgcttgaccctgagcctgtggaaatggcatagggaccagtctactacccactgggcctggtgtgtagagggggagagggtagcaaggtgcttctctacgcccatgacttgggagcaggtcttggcctccttcatgagagtctagtgccatgtcctgtcccatgatctggaccctgggactgtcttggcatcttaactgcagtttcaatgaggcagagggcaaagagagaccaagatcagaggggttcattatacccctggctagagaacccagctactgacatgcaagcagcttggggctggctggacacaggtactaggcccattgtttccaggtgaagctttcatcacagaacagtgttgtctccacctggccttagatggcacgccatgattcgggcctggatagactgcctgcgtccttaccactgatctggccaagaatgaggccctcccaacactttcactccctctccaagccttgatgggacctccacttatttaggcctcatgtgctttgaagaagctttgagagccaatgtgtcttccacgggtctcttttttgctacaagtaatcagccccatgtgttctcttaaactgagaattgcacctgggcaattcctgttttctaaggtggtctctgctgctatttaacaacccagagtaggcctctgtgaggcttcagtggcctcagaaaccagagggtccagatagggggcctgcttgggccctctgctgccaactgctcaaacctgctttagctccagccacttgtggcaaacaacctcgtttccttacaaattccagcatgtgactttggtgccgttacttgtgaaaaatctattctgttgtctttgatgtgtccaagaaaattcgtgtagtttacgtaaaaatatctgactcacaagaaagccaactgtatgtcttgtgatgggacagttcataatgtagttgctagaccactttacaaattgttcttgtcaccagatgtgttcagacattgctgtgcaattgttggggagggtagggggaaaggcgagaggagatacttattggtctttttgtttaataccttccccaagaggggacagtctggccaacttgctccagtaatgcaataaagacattgcaataaagtaaaaaaaaaaaaaaa

SEQ ID NO: 2 - protein, 461 aa, Homo sapiens
MYNTVWSMDRDDADWREVMMPYSTELIFYIEMDPPALPPKPPKPMTSAVPNGMKDSSVSLQDAEWYWGDISREEVNDKLRDMPDGTFLVRDASTKMQGDYTLTLRKGGNNKLIKIYHRDGKYGFSDPLTFNSVVELINHYHHESLAQYNPKLDVKLMYPVSRYQQDQLVKEDNIDAVGKKLQEYHSQYQEKSKEYDRLYEEYTRTSQEIQMKRTAIEAFNETIKIFEEQCHTQEQHSKEYIERFRREGNEKEIERIMMNYDKLKSRLGEIHDSKMRLEQDLKNQALDNREIDKKMNSIKPDLIQLRKIRDQHLVWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR